SEQUENCE LISTING (33 sequences)

SEQ ID NO: 1 - protein, 7 aa, Homo sapiens
DRVYIHP

SEQ ID NO: 2 - protein, 7 aa, Artificial Sequence
  Description of Artificial Sequence Synthetic peptide
DRVSIHC

SEQ ID NO: 3 - protein, 12 aa, Homo sapiens
DRVYIHPFHLVI

SEQ ID NO: 4 - protein, 7 aa, Artificial Sequence
  Description of Artificial Sequence Synthetic peptide
  MOD_RES (1)..(1): Any amino acid or dicarboxylic acid
  MOD_RES (2)..(2): Arg, Lys, Ala, Cit, Orn, acetylated Ser, Sar, D-Arg or D-Lys
  MOD_RES (3)..(3): Val, Ala, Leu, Nle, Ile, Gly, Lys, Pro, HydroxyPro, Aib, Acpc or Tyr
  MOD_RES (4)..(4): Tyr, Tyr(PO3), Thr, Ser, homoSer, azaTyr or Ala
  MOD_RES (5)..(5): Ile, Ala, Leu, norLeu, Val or Gly
  MOD_RES (6)..(6): His, Arg or 6-NH2-Phe
  MOD_RES (7)..(7): Cys, Pro or Ala
  see specification as filed for detailed description of substitutions and preferred embodiments
XXXXXXX

SEQ ID NO: 5 - protein, 7 aa, Artificial Sequence
  Description of Artificial Sequence Synthetic peptide
  MOD_RES (3)..(3): Nle
DRLYIHP

SEQ ID NO: 6 - protein, 7 aa, Artificial Sequence
  Description of Artificial Sequence Synthetic peptide
DRVSIHC

SEQ ID NO: 7 - protein, 7 aa, Artificial Sequence
  Description of Artificial Sequence Synthetic peptide
  MOD_RES (4)..(4): Any cross-linked amino acid
  misc_feature (4)..(7): Cross link between residues
  MOD_RES (7)..(7): Any cross-linked amino acid
DRVXIHX

SEQ ID NO: 8 - protein, 10 aa, Artificial Sequence
  Description of Artificial Sequence Synthetic peptide
  MOD_RES (3)..(3): Nle
  MOD_RES (4)..(4): Any cross-linked amino acid
  misc_feature (4)..(7): Cross link between residues
  MOD_RES (7)..(7): Any cross-linked amino acid
DRLXIHXFHL

SEQ ID NO: 9 - protein, 8 aa, Artificial Sequence
  Description of Artificial Sequence Synthetic peptide
  MOD_RES (3)..(3): Nle
  MOD_RES (4)..(4): Any cross-linked amino acid
  misc_feature (4)..(7): Cross link between residues
  MOD_RES (7)..(7): Any cross-linked amino acid
DRLXIHXF

SEQ ID NO: 10 - protein, 7 aa, Artificial Sequence
  Description of Artificial Sequence Synthetic peptide
  MOD_RES (2)..(2): Nle
  MOD_RES (3)..(3): Any cross-linked amino acid
  misc_feature (3)..(6): Cross link between residues
  MOD_RES (6)..(6): Any cross-linked amino acid
RLXIHXF

SEQ ID NO: 11 - protein, 6 aa, Artificial Sequence
  Description of Artificial Sequence Synthetic peptide
  MOD_RES (1)..(1): Nle
  MOD_RES (2)..(2): Any cross-linked amino acid
  misc_feature (2)..(5): Cross link between residues
  MOD_RES (5)..(5): Any cross-linked amino acid
LXIHXF

SEQ ID NO: 12 - protein, 7 aa, Artificial Sequence
  Description of Artificial Sequence Synthetic peptide
  MOD_RES (3)..(3): Nle
  MOD_RES (4)..(4): Any cross-linked amino acid
  misc_feature (4)..(7): Cross link between residues
  MOD_RES (7)..(7): Any cross-linked amino acid
DRLXIHX

SEQ ID NO: 13 - protein, 9 aa, Artificial Sequence
  Description of Artificial Sequence Synthetic peptide
  MOD_RES (3)..(3): Nle
  MOD_RES (4)..(4): Any cross-linked amino acid
  misc_feature (4)..(7): Cross link between residues
  MOD_RES (7)..(7): Any cross-linked amino acid
DRLXIHXFH

SEQ ID NO: 14 - protein, 7 aa, Artificial Sequence
  Description of Artificial Sequence Synthetic peptide
  MOD_RES (1)..(1): Any amino acid
  MOD_RES (2)..(2): Any positively-charged amino acid
  MOD_RES (3)..(3): Any aliphatic amino acid
  MOD_RES (4)..(4): Any cross-linked amino acid
  misc_feature (4)..(7): Thioether bridge between residues
  MOD_RES (5)..(5): Any aliphatic amino acid
  MOD_RES (7)..(7): Any cross-linked amino acid
XXXXXHX

SEQ ID NO: 15 - protein, 7 aa, Artificial Sequence
  Description of Artificial Sequence Synthetic peptide
  MOD_RES (4)..(4): Abu
DRVAIHA

SEQ ID NO: 16 - protein, 7 aa, Artificial Sequence
  Description of Artificial Sequence Synthetic peptide
DRVAIHA

SEQ ID NO: 17 - protein, 10 aa, Artificial Sequence
  Description of Artificial Sequence Synthetic peptide
  MOD_RES (1)..(1): Any amino acid or absent
  MOD_RES (2)..(2): Any positively charged amino acid or absent
  MOD_RES (3)..(3): Nle
  MOD_RES (4)..(4): Any cross-linked amino acid
  misc_feature (4)..(7): Thioether bridge between residues
  MOD_RES (5)..(5): Any aliphatic amino acid
  MOD_RES (7)..(7): Any cross-linked amino acid
  MOD_RES (8)..(8): Any amino acid other than Pro or absent
  misc_feature (9)..(9): May or may not be present
  MOD_RES (10)..(10): Any aliphatic residue or absent
  see specification as filed for detailed description of substitutions and preferred embodiments
XXLXXHXXHX

SEQ ID NO: 18 - protein, 7 aa, Artificial Sequence
  Description of Artificial Sequence Synthetic peptide
  MOD_RES (3)..(3): Nle
  MOD_RES (4)..(4): Abu
DRLAIHA

SEQ ID NO: 19 - protein, 7 aa, Artificial Sequence
  Description of Artificial Sequence Synthetic peptide
  MOD_RES (3)..(3): Nle
DRLAIHA

SEQ ID NO: 20 - protein, 8 aa, Artificial Sequence
  Description of Artificial Sequence Synthetic peptide
  MOD_RES (3)..(3): Nle
  MOD_RES (4)..(4): Abu
DRLAIHAI

SEQ ID NO: 21 - protein, 8 aa, Artificial Sequence
  Description of Artificial Sequence Synthetic peptide
  MOD_RES (3)..(3): Nle
DRLAIHAI

SEQ ID NO: 22 - protein, 7 aa, Artificial Sequence
  Description of Artificial Sequence Synthetic peptide
  misc_feature (4)..(7): Cross link between residues
DRVSIHC

SEQ ID NO: 23 - protein, 8 aa, Homo sapiens
DRVYIHPF

SEQ ID NO: 24 - protein, 9 aa, Homo sapiens
DRVYIHPFH

SEQ ID NO: 25 - protein, 10 aa, Homo sapiens
DRVYIHPFHL

SEQ ID NO: 26 - protein, 6 aa, Homo sapiens
RVYIHP

SEQ ID NO: 27 - protein, 7 aa, Homo sapiens
RVYIHPF

SEQ ID NO: 28 - protein, 8 aa, Homo sapiens
RVYIHPFH

SEQ ID NO: 29 - protein, 9 aa, Homo sapiens
RVYIHPFHL

SEQ ID NO: 30 - protein, 6 aa, Homo sapiens
VYIHPF

SEQ ID NO: 31 - protein, 7 aa, Homo sapiens
VYIHPFH

SEQ ID NO: 32 - protein, 8 aa, Homo sapiens
VYIHPFHL

SEQ ID NO: 33 - protein, 7 aa, Artificial Sequence
  Description of Artificial Sequence Synthetic peptide
  misc_feature (4)..(7): Cross link between residues
  see specification as filed for detailed description of substitutions and preferred embodiments
DRVYIHP

(19) United States
(12) Patent Application Publication
(10) Pub. No.: US 2018/0221433 A1
(43) Pub. Date: Aug. 9, 2018

(54) ANGIOTENSINS IN MUSCULAR DYSTROPHY

(71) Applicants: University of Iowa Research Foundation, Lowa City, IA (US); Tarix Orphan, LLC, Cambridge, MA (US)

(72) Inventors: Rasna Sabharwal, Iowa City, IA (US); Mark Chapleau, Iowa City, IA (US); Richard Franklin, Cambridge, MA (US)

(21) Appl. No.: 15/695,731

(22) Filed: Sep. 5, 2017

Related U.S. Application Data
(63) Continuation of application No. 14/785,538, filed on Oct. 19, 2015, now abandoned.
(60) Provisional application No. 61/813,929, filed on Apr. 19, 2013.
(60) Provisional application No. 61/818,307, filed on May 1, 2013.

(51) Int. Cl. A61K 38/08; A61K 31/4178; A61K 45/06

(52) U.S. Cl. CPC A61K 38/085; A61K 31/4178; A61K 2300/00; A61K 45/06

(57) ABSTRACT

The present invention provides, among other things, methods of treating a muscular dystrophy including administering to a subject suffering from or susceptible to a muscular dystrophy an angiotensin (1-7) peptide. The present invention is, in part, based on the unexpected discovery that administration of an angiotensin (1-7) peptide in a muscular dystrophy animal model reduces fibrosis, restores locomotor activity and restores sympathovagal balance, which are characteristic symptoms in patients suffering from muscular dystrophy. Thus, the present invention provides a new and more effective therapy for muscular dystrophy. In some embodiments, an angiotensin (1-7) peptide includes the naturally occurring angiotensin (1-7) amino acid sequence of Asp1-Arg2-Val3-Tyr4-Ile5-His6-Pro7 (SEQID NO:1). In some embodiments, the angiotensin (1-7) peptide is a functional equivalent of SEQ ID NO:1. In some embodiments, the linear peptide has an amino acid sequence of Asp1-Arg2-Val3-Ser4-Ile5-H′is6-Cys7 (SEQ ID NO:2). In some embodiments, the cyclic peptide is a 4,7-cyclized angiotensin (1-7).

### CROSS-REFERENCE TO RELATED APPLICATIONS

This application is a continuation application of U.S. patent application Ser. No. 14/785,538, filed on Oct. 19, 2015, which is a national stage application filed under 35 U.S.C. § 371, of international application serial no. PCT/US14/34615, filed on Apr. 18, 2014, which claims priority from U.S. provisional patent application Ser. No. 61/813,929, filed Apr. 19, 2013 and U.S. provisional patent application Ser. No. 61/818,307, filed May 1, 2013, all the disclosures of which are hereby incorporated by reference in their entirety.

### STATEMENT REGARDING FEDERALLY SPONSORED RESEARCH

This invention was made with government support under HL14388 awarded by National Institutes of Health (NIH). The government has certain rights in the invention.

### INCORPORATION-BY-REFERENCE-OF SEQUENCE LISTING

The contents of the file named “TRX-002US2_SL.txt”, which was created on Oct. 19, 2015 and is 11 KB in size, are hereby incorporated by reference in their entirety.

### BACKGROUND

Muscular Dystrophy (MD) refers to a group of diseases in which muscle fibers are abnormally susceptible to damage. Some subtypes, like Duchenne Muscular Dystrophy, typically manifest very early in life, while others, such as Opthalmoplegic Muscular Dystrophy tend to manifest much later in life. Current treatments for muscular dystrophy include corticosteroid administration, use of orthopaedic devices to support locomotion and prevent contractures, and physiotherapy. Unfortunately, none of the currently used treatments have proven capable of arresting or reversing the progression of the disease.

### SUMMARY OF THE INVENTION

The present invention provides an improved method of treating muscular dystrophy based on angiotensin(1-7) peptides or angiotensin (1-7) receptor agonists. The present invention is, in part, based on the unexpected discovery that administration of an angiotensin (1-7) peptide in a muscular dystrophy animal model reduces fibrosis, restores locomotor activity and restores sympathovagal balance, which are characteristic symptoms in patients suffering from muscular dystrophy. Thus, the present invention provides a new and more effective therapy for muscular dystrophy.

In one aspect, the present invention provides methods of treating a muscular dystrophy including administering to a subject suffering from or susceptible to a muscular dystrophy an angiotensin (1-7) peptide and/or angiotensin (1-7) receptor agonist. In some embodiments, the angiotensin (1-7) peptide and/or angiotensin (1-7) receptor agonist is administered at an effective dose periodically at an administration interval such that at least one symptom or feature of a muscular dystrophy is reduced in intensity, severity, duration, or frequency or has delayed in onset. In some embodiments, the at least one symptom or feature of muscular dystrophy is selected from the group consisting of muscle wasting, muscle weakness, muscle fragility, muscle pseudohypertrophy, joint contracture, skeletal deformation, cardiomyopathy, impaired swallowing, impaired bowel and bladder function, muscle ischemia, cognitive impairment, behavioral dysfunction, socialization impairment, scoliosis, and impaired respiratory function.

In some embodiments, the administration of the angiotensin (1-7) peptide and/or angiotensin (1-7) receptor agonist results in muscle regeneration, fibrosis reduction, increased muscle strength, increased flexibility, increased range of motion, increased stamina, reduced fatiguability, increased blood flow, improved cognition, improved pulmonary function, and/or inflammation inhibition.

In some embodiments, the muscular dystrophy is selected from the group consisting of Duchenne muscular dystrophy, Becker's muscular dystrophy, Becker myotonia congenita, Emery-Dreifuss muscular dystrophy, Miyoshi myopathy, Congenital muscular dystrophy, Facioscapulohumeral muscular dystrophy, Oculopharyngeal muscular dystrophy, Primary lateral sclerosis, Spinal muscular atrophy, polymyositis, Guillian-Barre Syndrome, Anderson-Tawil syndrome, Bethlem myopathy, Bulbospinal muscular atrophy, Carnitine deficiency, Carnitine palmityl transferase deficiency, Central core disease, Cantronuclear myopathy, Charcot-marie-tooth disease, Congenital myasthenic syndromes, Congenital myotonic dystrophy (Walker-Warburg Syndrome), Cori disease (Debrancher enzyme deficiency), Dejerine-Sottas Disease, dermatomyositis, distal muscular dystrophy, Dystrophia myotonica (Myotonic muscular dystrophy), Endocrine myopathies, Eulenberg disease (Paramyotonia congenital), Finnish distal myopathy, Forbes disease, Friedrich's ataxia, Fukuyama congenital muscular dystrophy, Glycogenosis Types 2, 3, 5, 7, 9, 10, 11, Gowers-Laing distal myopathy, Heredity inclusion-body myositis, Hyperthyroid myopathy, Hypothyroid myopathy, Inclusion-Body myositis, Inherited myopathies, Integrin-deficient congenital muscular dystrophy, Kennedy disease (Spinal-Bulbar muscular atrophy), Lactate dehydrogenase deficiency, Lambert-Eaton myasthenic syndrome, limb-girdle muscular dystrophy, McArdle disease, Merosin-deficient congenital muscular dystrophy, Mitochondrial myopathy, Motor neurone disease, Muscle-eye-brain disease, Myasthenia gravis, Myoadenylate deaminase deficiency, myofibrillar myopathy, Myophosphorylase deficiency, Myotonia congenita (Thomsen disease), Myotubular myopathy, Myotonic muscular dystrophy, Nemaline myopathy, Nonaka distal myopathy, Paramyotonia congenital, Pearson syndrome, Periodic paralysis, Phosphofructokinase deficiency (Tarui disease), Phosphoglycerate kinase deficiency, Phosphoglycerate Mutase deficiency, Phosphorylase deficiency, Pompe disease (acid maltase deficiency), Progressive external ophthalmoplegia, Welander distal myopathy, and ZASP-related myopathy. In some embodiments, the congenital muscular dystrophy is selected from the group consisting of laminin-α2-deficient congenital muscular dystrophy, Ullrich congenital muscular dystrophy, Walker-Warburg syndrome, Fukuyama congenital muscular dystrophy, and congenital muscular dystrophy with mental retardation and pachygyria. In some embodiments, the muscular dystrophy is Duchenne muscular dystrophy.

Any of a variety of routes of administration may be used according to various embodiments. In some embodiments, an angiotensin (1-7) peptide and/or angiotensin (1-7) receptor agonist is administered parenterally. In some embodiments, the parenteral administration is selected from intravenous, intradermal, inhalation, transdermal (topical), intraocular, intramuscular, subcutaneous, intramuscular, and/or transmucosal administration. In some embodiments, an angiotensin (1-7) peptide and/or angiotensin (1-7) receptor agonist is administered orally.

In some embodiments, an angiotensin (1-7) peptide and/or angiotensin (1-7) receptor agonist is administered weekly, daily, or at variable intervals. In some embodiments, an angiotensin (1-7) peptide and/or angiotensin (1-7) receptor agonist is administered at an effective dose ranging from about 1-1,000 μg/kg/day. In some embodiments, an angiotensin (1-7) peptide and/or angiotensin (1-7) receptor agonist is administered at an effective dose ranging from about 50-500 μg/kg/day. In some embodiments, an angiotensin (1-7) peptide and/or angiotensin (1-7) receptor agonist is administered at an effective dose ranging from about 400-500 μg/kg/day.

In some embodiments, an angiotensin (1-7) peptide and/or angiotensin (1-7) receptor agonist is administered in combination with an anti-muscular dystrophy medication. In some embodiments, the anti-muscular dystrophy medication is selected from the group consisting of Eteplirsen (AVI-4658), HCT 1026, NCX 320, sildenafil, tadalafil, vardenafil, avanafil, iodenafil, mirodenafil, udenafil, zaprinast, a corticosteroid, and combinations thereof.

In some embodiments, an angiotensin (1-7) peptide includes the naturally-occurring Angiotensin (1-7) amino acid sequence of Asp¹-Arg²-Val³-Tyr⁴-Ile⁵-His⁶-Pro⁷ (SEQ ID NO:1). In some embodiments, the angiotensin (1-7) peptide is a functional equivalent of SEQ ID NO:1.

In some embodiments, the functional equivalent is a linear peptide. In some embodiments, the linear peptide includes a sequence that includes at least four amino acids from the seven amino acids that appear in the naturally-occurring Angiotensin (1-7), wherein the at least four amino acids maintain their relative positions as they appear in the naturally-occurring Angiotensin (1-7). In some embodiments, the linear peptide contains 4-25 amino acids. In some embodiments, the linear peptide is a fragment of the naturally-occurring Angiotensin (1-7). In some embodiments, the linear peptide contains amino acid substitutions, deletions and/or insertions in the naturally-occurring Angiotensin (1-7). In some embodiments, the linear peptide has an amino acid sequence of Asp¹-Arg²-Val³-Ser⁴-Ile⁵-His⁶-Cys⁷ (SEQ ID NO:2).

In some embodiments, the functional equivalent is a cyclic peptide. In some embodiments, the cyclic peptide includes a linkage between amino acids. In some embodiments, the linkage is located at residues corresponding to positions Tyr⁴ and Pro⁷ in naturally-occurring Angiotensin (1-7). In some embodiments, the linkage is a thioether bridge. In some embodiments, the cyclic peptide includes an amino acid sequence otherwise identical to the naturally-occurring Angiotensin (1-7) amino acid sequence of Asp¹-Arg²-Val³-Tyr⁴-Ile⁵-His⁶-Pro⁷ (SEQ ID NO:1). In some embodiments, the cyclic peptide is a 4,7-cyclized angiotensin (1-7) with the following formula:

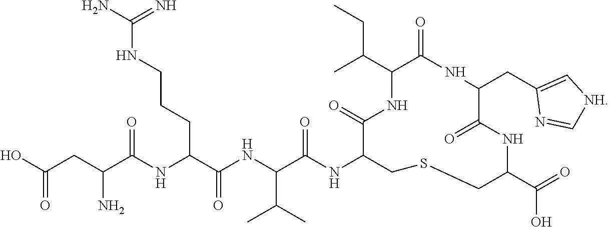 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In some embodiments, the angiotensin (1-7) peptide comprises one or more chemical modifications to increase protease resistance, serum stability and/or bioavailability. In some embodiments, the one or more chemical modifications comprise pegylation.

In some embodiments, an angiotensin (1-7) receptor agonist is used to treat muscular dystrophy according to the present invention. In some embodiments, the angiotensin (1-7) receptor agonist is a non-peptidic agonist. In some embodiments, the non-peptidic agonist is a compound with the following structure:

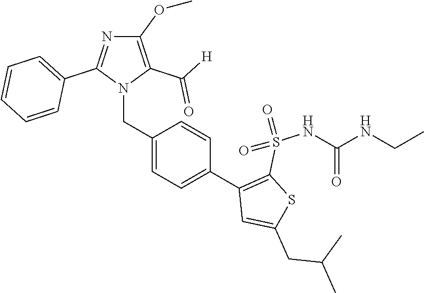 [SMILES: CCNC(=O)NS(=O)(=O)c1sc(CC(C)C)cc1-c1ccc(Cn2c(-c3ccccc3)nc(OC)c2C=O)cc1]

or a pharmaceutically acceptable salt thereof. In some embodiments, an angiotensin (1-7) receptor agonist is administered orally.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### Definitions

In order for the present invention to be more readily understood, certain terms are first defined below. Additional definitions for the following terms and other terms are set forth throughout the specification.

Agonist: As used herein, the term “agonist” refers to any molecule that has a positive impact in a function of a protein of interest. In some embodiments, an agonist directly or indirectly enhances, strengthens, activates and/or increases an activity of a protein of interest. In particular embodiments, an agonist directly interacts with the protein of interest. Such agonists can be, e.g., proteins, chemical compounds, small molecules, nucleic acids, antibodies, drugs, ligands, or other agents.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally-occurring angiotensin compounds is considered to be biologically active.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Functional equivalent or derivative: As used herein, the term “functional equivalent” or “functional derivative” denotes a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of disease as the individual being treated, who is about the same age as the individual being treated (to ensure that the stages of the disease in the treated individual and the control individual(s) are comparable).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Muscular Dystrophy: As used herein, the term “muscular dystrophy” or “MD”, refers to a group of diseases that weaken the musculoskeletal system. In some embodiments, a muscular dystrophy is an MD-like condition. In some embodiments, such diseases include progressive wasting of skeletal muscle. In some embodiments, cardiac and smooth muscle are affected.

Prevent: As used herein, the term “prevent” or “prevention”, when used in connection with the occurrence of a disease, disorder, and/or condition, refers to reducing the risk of developing the disease, disorder and/or condition. See the definition of “risk.”

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Risk: As will be understood from context, a “risk” of a disease, disorder, and/or condition comprises a likelihood that a particular individual will develop a disease, disorder, and/or condition (e.g., a muscular dystrophy). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., a muscular dystrophy). In some embodiments a reference sample or group of reference samples are from individuals comparable to a particular individual. In some embodiments, relative risk is 0, 1, 2, 3, 4, 5, 6, 7, 8, 9, 10, or more.

Stability: As used herein, the term “stable” refers to the ability of the therapeutic agent to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In certain embodiments, pharmaceutical compositions described herein have been formulated such that they are capable of stabilizing, or alternatively slowing or preventing the degradation, of one or more therapeutic agents formulated therewith. In the context of a formulation a stable formulation is one in which the therapeutic agent therein essentially retains its physical and/or chemical integrity and biological activity upon storage and during processes (such as freeze/thaw, mechanical mixing and lyophilization).

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, condition, or event (for example, Muscular Dystrophy) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event (5) having undergone, planning to undergo, or requiring a transplant. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### BRIEF DESCRIPTION OF THE DRAWING

FIG. 1A shows several schematic views of the telemetry used in Example 2 along with some of the analyses conducted, and FIG. 1B shows the design of the experiment shown in Example 2.

FIG. 2A shows bar graphs depicting mean arterial blood pressure between control C57BL6 mice +/− treatment with Ang (1-7) and Sgcd −/− mice +/− treatment with Ang (1-7), and FIG. 2B shows 24 hour average heart rate between control C57BL6 mice +/− treatment with Ang (1-7) and Sgcd −/− mice +/− treatment with Ang (1-7).

FIG. 3 shows a bar graph representing 24 hour average locomotor activity between control C57BL6 mice +/− treatment with Ang (1-7) and Sgcd −/− mice +/− treatment with Ang (1-7), as measured via radiotelemetry.

FIG. 4A shows bar graphs depicting baroreflex sensitivity between control C57BL6 mice +/− treatment with Ang (1-7) and Sgcd −/− mice +/− treatment with Ang (1-7), FIG. 4B shows cardiac vagal tone as measured by response to atropine between control C57BL6 mice +/− treatment with Ang (1-7) and Sgcd −/− mice +/− treatment with Ang (1-7), FIG. 4C shows cardiac sympathetic tone as measured by response to propranolol between control C57BL6 mice +/− treatment with Ang (1-7) and Sgcd −/− mice +/− treatment with Ang (1-7), and FIG. 4D shows vasomotor sympathetic tone as measured by response to chlorisondamine, between control C57BL6 mice +/− treatment with Ang (1-7) and Sgcd −/− mice +/− treatment with Ang (1-7).

FIG. 5A shows histopathological data using Masson Trichrome stain to show a comparison of fibrosis of muscle tissue between control C57BL6 mice +/− treatment with Ang (1-7) and Sgcd −/− mice +/− treatment with Ang (1-7), and FIG. 5B shows a bar graph quantitating the results in panel A.

FIG. 6 displays the results of Alexa Fluor 488 staining showing the levels of angiotensin type 1 receptors (AT₁R) receptor expression in the tissues of control C57BL6 mice +/− treatment with Ang (1-7) and Sgcd −/− mice +/− treatment with Ang (1-7).

FIG. 7A shows: dihydroethidium (DHE) staining of skeletal muscle cells in control C57BL6 mice +/− treatment with Ang (1-7) and Sgcd −/− mice +/− treatment with Ang (1-7), and FIG. 7B shows a bar graph representing the data shown in panel A.

FIG. 8 shows an exemplary graph of the average revolutions per minute made by Sgcd −/− mice on a running-wheel during the sixth, seventh, and eighth weeks of treatment with one of: saline, TXA127 (SEQ ID NO: 1), Pancyte (SEQ 10 NO: 22), or TXA 301 (SEQ ID NO: 2). Data=means±SE, and *=p<0.0.5 vs. saline group.

FIG. 9 shows an exemplary graph of the average revolutions per minute on a running wheel made by Sgcd −/− mice treated with one of: saline, TXA127 (SEQ ID NO: 1), Pancyte (SEQ ID NO: 22), or TXA 301 (SEQ ID NO: 2) for 20 days.

FIG. 10A shows exemplary Hematoxylin-Eosin stains (top row) and Masson's Trichrome stain (bottom row) of quadriceps muscle taken from Sgcd −/− mice between 36-43 weeks of age that were treated with one of: saline, TXA127 (SEQ ID NO: 1), Pancyte (SEQ IDNO: 22), or TXA 301 (SEQ ID NO: 2); FIG. 10B shows the amount of fibrosis calculated from the Masson's Trichrome data (calculated as area of the blue color) and is represented as a bar graph. Data=means±SE, and *=p<0.05 vs. saline group.

FIG. 11A shows exemplary confocal images of quadriceps muscles taken from Sgcd −/− mice treated, with one of: saline, TXA127 (SEQ ID NO:1), Pancyte (SEQ ID NO: 22), or TXA301 (SEQ ID NO: 2), with oxidative stress indicated through a dihydroethidium (DHE) stain; FIG. 11B shows a bar graph depicting an average of six sections of stained quadriceps after the intensity of fluorescence was quantified using NIH ImageJ software and normalized to a percentage of the average fluorescence intensity measured in saline-treated Sgcd −/− mice. Data=means±SE, and *=p<0.05 vs. saline group.

### DETAILED DESCRIPTION OF CERTAIN EMBODIMENTS

The present invention provides, among other things, improved compositions and methods based on the use of angiotensin (1-7) peptides, analogs or derivatives thereof, or angiotensin (1-7) receptor agonists like A VE0991 for treating a muscular dystrophy. In some embodiments, at least one symptom or feature of a muscular dystrophy is reduced in intensity, severity, duration, or frequency or has delayed in onset. In some embodiments, the administration of one or more angiotensin (1-7) peptides, analogs or derivatives thereof, or angiotensin (1-7) receptor agonist results in muscle regeneration, fibrosis reduction, increased muscle strength, increased flexibility, increased range of motion, increased stamina, reduced fatiguability, increased blood flow, improved cognition, improved pulmonary function, and/or inflammation inhibition.

Various aspects of the invention are described in detail in the following sections. The use of sections is not meant to limit the invention. Each section can apply to any aspect of the invention. In this application, the use of “or” means “and/or” unless stated otherwise.

### Muscular Dystrophy

Muscular dystrophies (MD) are a group of inherited disorders that cause degeneration of muscle, leading to weak and impaired movements. A central feature of all muscular dystrophies is that they are progressive in nature. Muscular dystrophies include, but are not limited to: Duchenne muscular dystrophy, Becker's muscular dystrophy, Becker myotonia congenita, Emery-Dreifuss muscular dystrophy, Miyoshi myopathy, Congenital muscular dystrophy, acioscapulohumeral muscular dystrophy, Oculopharyngeal muscular dystrophy, Primary lateral sclerosis, Spinal muscular atrophy, polymyositis, Guillian-Barre Syndrome, Anderson-Tawil syndrome, Bethlem myopathy, Bulbospinal muscular atrophy, Carnitine deficiency, Carnitine palmityl transferase deficiency, Central core disease, Cantronuclear myopathy, Charcot-marie-tooth disease, Congenital myasthenic syndromes, Congenital myotonic dystrophy (Walker-Warburg Syndrome), Cori disease (Debrancher enzyme deficiency), Dejerine-Sottas Disease, dermatomyositis, distal muscular dystrophy, Dystrophia myotonica (Myotonic muscular dystrophy), Endocrine myopathies, Eulenberg disease (Paramyotonia congenital), Finnish distal myopathy, Forbes disease, Friedrich's ataxia, Fukuyama congenital muscular dystrophy, Glycogenosis Types 2, 3, 5, 7, 9, 10, 11, Gowers-Laing distal myopathy, Heredity inclusion-body myositis, Hyperthyroid myopathy, Hypothyroid myopathy, Inclusion-Body myositis, Inherited myopathies, Integrin-deficient congenital muscular dystrophy, Kennedy disease (Spinal-Bulbar muscular atrophy), Lactate dehydrogenase deficiency, Lambert-Eaton myasthenic syndrome, McArdle disease, Merosin-deficient congenital muscular dystrophy, Mitochondrial myopathy, Motor neurone disease, Muscle-eye-brain disease, Myasthenia gravis, Myoadenylate deaminase deficiency, myofibrillar myopathy, Myophosphorylase deficiency, Myotonia congcnita (Thomsen disease), Myotubular myopathy, Myotonic muscular dystrophy, Nemaline myopathy, Nonaka distal myopathy, Paramyotonia congenital, Pearson syndrome, Periodic paralysis, Phosphofructokinase deficiency (Tarui disease), Phosphoglycerate kinase deficiency, Phosphoglycerate Mutase deficiency, Phosphorylase deficiency, Pompe disease (acid maltase deficiency), Progressive external ophthalmoplegia, Welander distal myopathy, and ZASP-related myopathy. In some embodiments, the congenital muscular dystrophy is selected from the group consisting of laminin-α2-deficient congenital muscular dystrophy, Ullrich congenital muscular dystrophy, Walker-Warburg syndrome, Fukuyama congenital muscular dystrophy, and congenital muscular dystrophy with mental retardation and pachygyria. In some embodiments, the muscular dystrophy is Duchenne muscular dystrophy.

Symptoms of muscular dystrophy may vary by type of muscular dystrophy with some or all muscles being affected. Exemplary symptoms of muscular dystrophies include delayed development of muscle motor skills, difficulty using one or more muscle groups, muscle wasting, muscle weakness, muscle fragility, muscle pseudohypertrophy, joint contracture, skeletal deformation, cardiomyopathy, muscle ischemia, scoliosis, difficulty swallowing, speaking or eating, impaired bowel and bladder function, drooling, eyelid drooping, frequent falling, loss of strength in a muscle or group of muscles as an adult, loss in muscle size, problems walking due to weakness or altered biomechanics of the body, impaired respiratory function, and/or cognitive or behavioral impairment/mental retardation.

While there are no known cures for muscular dystrophies, several supportive treatments are used which include both symptomatic and disease modifying therapies. Corticosteroids, ACE inhibitors, Angiotensin receptor blockers, physical therapy, orthotic devices, wheelchairs, or other assistive medical devices for ADLs and pulmonary function are commonly used in muscular dystrophies. Cardiac pacemakers are used to prevent sudden death from cardiac arrythmias in Myotonic dystrophy. Anti-myotonic agents which improve the symptoms of myotonia (inability to relax) include mexilitine, and in some cases phenytoin, procainamide and quinine.

Duchenne Muscular Dystrophy

Duchenne muscular dystrophy (DMD) is a recessive X-linked form of muscular dystrophy which results in muscle degeneration and eventual death. DMD is characterized by weakness in the proximal muscles, abnormal gait, hypertrophy in the gastrocnemius (calf) muscles, and elevated creatine kinase. Many DMD patients are diagnosed around the age of 5, when symptoms/signs typically become more obvious. Affected individuals typically stop walking around age 10-13 and die in or before their mid to late 20's due to cardiorespiratory dysfunction.

The disorder DMD is caused by a mutation in the dystrophin gene, located on the human X chromosome, which codes for the protein dystrophin, an important structural component within muscle tissue that provides structural stability to the dystroglycan complex (DGC) of the cell membrane. Dystrophin links the internal cytoplasmic actin filament network and extracellular matrix, providing physical strength to muscle fibers. Accordingly, alteration or absence of dystrophin results in abnormal sarcolemnal membrane tearing and necrosis of muscle fibers. While both sexes can carry the mutation, females rarely exhibit severe signs of the disease.

A main symptom of DMD is muscle weakness associated with muscle wasting with the voluntary muscles being affected first typically, especially affecting the muscles of the hips, pelvic area, thighs, shoulders, and calf muscles. Muscle weakness also occurs in the arms, neck, and other areas. Calves are often enlarged. Signs and symptoms usually appear before age 6 and may appear as early as infancy. Other physical symptoms include, but are not limited to, delayed ability to walk independently, progressive difficulty in walking, stepping, or running, and eventual loss of ability to walk (usually by the age of 12), frequent falls, fatigue; difficulty with motor skills (running, hopping, jumping), increased lumbar lordosis, leading to shortening of the hip-flexor muscles, contractures of Achilles tendon and hamstrings impairing functionality because the muscle fibers shorten and fibrosis occurs in connective tissue, muscle fiber deformities, pseudohypertrophy (enlarging) of tongue and calf muscles caused by replacement of muscle tissue by fat and connective tissue, higher risk of neurobehavioral disorders (e.g., ADHD), learning disorders (dyslexia), and non-progressive weaknesses in specific cognitive skills (in particular short-term verbal memory), skeletal deformities (including scoliosis in some cases).

Becker's Muscular Dystrophy

Becker muscular dystrophy is an X-linked recessive inherited disease characterized by a progressive weakening of the muscles of the legs and pelvis. Like DMD, Becker's muscular dystrophy is caused by a mutation in the dystrophin gene, however, unlike DMD, a subject suffering from Becker's muscular dystrophy generally produces a higher level of functional dystrophin than a sufferer of DMD, who may produce practically no functional dystrophin. As a result, Becker's muscular dystrophy is typically thought of as less severe than DMD.

Symptoms of Becker's muscular dystrophy include progressive muscle weakness, particularly in the lower extremities, toe-walking, frequent falls, difficulty breathing, skeletal deformities such as scoliosis, fatigue, and elevated creatine phosphokinase (CPK). The severity and array of symptoms varies from individual to individual, to a much larger extent than in DMD.

Limb-Girdle Muscular Dystrophy

While limb-girdle muscular dystrophy bears some similarity to both Duchenne and Becker's muscular dystrophy, it presents a distinct pathology most greatly affecting the muscles of the hip and shoulder (i.e., the “limb-girdle” muscles). While the precise mechanism underlying limb-girdle muscular dystrophy is not known, it is thought the several sarcoglycans including α, β, γ, and δ sarcoglycans are thought to play a significant role in the development and progression of the disease.

Given the course of development in limb-girdle muscular dystrophy, it may often not be fatal (though failure of heart and respiratory muscles can happen as in the most severe form of limb-girdle muscular dystrophy, known as LGMD2C). However, it is typical for a subject to be confined to a wheelchair within 20 years of diagnosis with the disease. While some symptoms of limb-girdle muscular dystrophy overlap with other types of muscular dystrophy, symptoms particularly common to sufferers of limb-girdle MD include: difficulty walking, difficulty bending over, and falling while walking or climbing stairs.

Angiotensin (1-7) Peptides and/or Angiotensin (1-7) Receptor Agonists

The present invention provides, among other things, methods of treating a muscular dystrophy including administering to a subject suffering from or susceptible to a muscular dystrophy an angiotensin (1-7) peptide.

Angiotensin (1-7) Peptides

As used herein, the term “angiotensin (1-7) peptide” refers to both naturally-occurring Angiotensin (1-7) and any functional equivalent, analogue or derivative of naturally-occurring Angiotensin (1-7). As used herein, “peptide” and “polypeptide” are interchangeable terms and refer to two or more amino acids bound together by a peptide bond. As used herein, the terms “peptide” and “polypeptide” include both linear and cyclic peptide. The terms “angiotensin-(1-7)”, “Angiotensin-(1-7)”, “Ang-(1-7)”, and “TXA-127” are used interchangeably.

Naturally-Occurring Angiotensin (1-7)

Naturally-occurring Angiotensin (1-7) (also referred to as Ang-(1-7)) is a seven amino acid peptide shown below:

(SEQ ID NO: 1)
Asp¹-Arg²-Val³-Tyr⁴-Ile⁵-His⁶-Pro⁷

It is part of the renin-angiotensin system and is converted from a precursor, also known as Angiotensinogen, which is an α-2-globulin that is produced constitutively and released into the circulation mainly by the liver. Angiotensinogen is a member of the serpin family and also known as renin substrate. Human angiotensinogen is 452 amino acids long, but other species have angiotensinogen of varying sizes. Typically, the first 12 amino acids are the most important for angiotensin activity:

(SEQ ID NO: 3)
Asp¹-Arg²-Val³-Tyr⁴-Ile⁵-His⁶-Pro⁷-Phe⁸-His⁹-
Leu¹⁰-Val¹¹-Ile¹²

Different types of angiotensin may be formed by the action of various enzymes. For example, Angiotensin (1-7) is generated by action of Angiotensin-converting enzyme 2 (ACE 2).

Ang-(1-7) is an endogenous ligand for Mas receptors. Mas receptors are G-protein coupled receptor containing seven transmembrane spanning regions. As used herein, the term “angiotensin-(1-7) receptor’ encompasses the G Protein-Coupled Mas Receptors.

As used herein, the term “naturally-occurring Angiotensin (1-7)” includes any Angiotensin (1-7) peptide purified from natural sources and any recombinantly produced or chemically synthesized peptides that have an amino acid sequence identical to that of the naturally-occurring Angiotensin (1-7).

Functional Equivalents, Analogs or Derivatives of Ang-(1-7)

In some embodiments, an angiotensin (1-7) peptide suitable for the present invention is a functional equivalent of naturally-occurring Ang-(1-7). As used herein, a functional equivalent of naturally-occurring Ang-(1-7) refers to any peptide that shares amino acid sequence identity to the naturally-occurring Ang-(1-7) and retain substantially the same or similar activity as the naturally-occurring Ang-(1-7). For example, in some embodiments, a functional equivalent of naturally-occurring Ang-(1-7) described herein has pro-angiogenic activity as determined using methods described herein or known in the art, or an activity such as nitric oxide release, vasodilation, improved endothelial function, antidiuresis, or one of the other properties discussed herein, that positively impacts angiogenesis. In some embodiments, a functional equivalent of naturally-occurring Ang-(1-7) described herein can bind to or activate an angiotensin-(1-7) receptor (e.g., the G protein-coupled Mas receptor) as determined using various assays described herein or known in the art. In some embodiments, a functional equivalent of Ang-(1-7) is also referred to as an angiotensin (1-7) analogue or derivative, or functional derivative.

Typically, a functional equivalent of angiotensin (1-7) shares amino acid sequence similarity to the naturally-occurring Ang-(1-7). In some embodiments, a functional equivalent of Ang-(1-7) according to the invention contains a sequence that includes at least 3 (e.g., at least 4, at least 5, at least 6, at least 7) amino acids from the seven amino acids that appear in the naturally-occurring Ang-(1-7), wherein the at least 3 (e.g., at least 4, at least 5, at least 6, or at least 7) amino acids maintain their relative positions and/or spacing as they appear in the naturally-occurring Ang-(1-7).

In some embodiments, a functional equivalent of Ang-(1-7) also encompasses any peptide that contains a sequence at least 50% (e.g., at least 60%, 70%, 80%, or 90%) identical to the amino acid sequence of naturally-occurring Ang-(1-7). Percentage of amino acid sequence identity can be determined by alignment of amino acid sequences. Alignment of amino acid sequences can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). W U-BLAST-2 uses several search parameters, most of which are set to the default values. The adjustable parameters are set with the following values: overlap span=1, overlap fraction=0.125, word threshold (T)=11. HSP score (S) and HSP S2 parameters are dynamic values and are established by the program itself, depending upon the composition of the particular sequence, however, the minimum values may be adjusted and are set as indicated above.

In some embodiments, a functional equivalent, analogue or derivative of Ang-(1-7) is a fragment of the naturally-occurring Ang-(1-7). In some embodiments, a functional equivalent, analogue or derivative of Ang-(1-7) contains amino acid substitutions, deletions and/or insertions in the naturally-occurring Ang-(1-7). Ang-(1-7) functional equivalents, analogues or derivatives can be made by altering the amino acid sequences by substitutions, additions, and/or deletions. For example, one or more amino acid residues within the sequence of the naturally-occurring Ang-(1-7) (SEQ ID NO:1) can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophan, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. For example, the amino acid sequence of a peptide inhibitor can be modified or substituted.

Examples of Ang-(1-7) functional equivalents, analogues and derivatives are described in the section entitled “Exemplary Angiotensin(1-7) Peptides” below.

An angiotensin-(1-7) peptide can be of any length. In some embodiments, an angiotensin-(1-7) peptide according to the present invention can contain, for example, from 5-25 amino acid residues, such as 5-20, 5-15 or 5-10 amino acid residues. In some embodiments, an Ang-(1-7) peptide according to the present invention contain 5, 6, 7, 8, 9, 10, 11, 12, 13, 14, 15, 16, 17, 18, 19, 20, 21, 22, 23, 24 or 25 residues.

In some embodiments, an angiotensin-(1-7) peptide contains one or more modifications to increase protease resistance, serum stability and/or bioavailability. In some embodiments, suitable modifications are selected from pegylation, acetylation, glycosylation, biotinylation, substitution with D-amino acid and/or un-natural amino acid, and/or cyclization of the peptide.

As used herein, the term “amino acid,” in its broadest sense, refers to any compound and/or substance that can be incorporated into a polypeptide chain. In certain embodiments, an amino acid has the general structure H₂N—C(H)(R)—COOH. In certain embodiments, an amino acid is a naturally-occurring amino acid. In certain embodiments, an amino acid is a synthetic or un-natural amino acid (e.g., α,α-disubstituted amino acids, N-alkyl amino acids); in some embodiments, an amino acid is a D-amino acid; in certain embodiments, an amino acid is an L-amino acid. “Standard amino acid” refers to any of the twenty standard amino acids commonly found in naturally occurring peptides including both L- and D-amino acids which are both incorporated in peptides in nature. “Nonstandard” or “unconventional amino acid” refers to any amino acid, other than the standard amino acids, regardless of whether it is prepared synthetically or obtained from a natural source. As used herein, “synthetic or un-natural amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting its activity. Examples of unconventional or un-natural amino acids include, but are not limited to, citrulline, ornithine, norleucine, norvaline, 4-(E)-butenyl-4(R)-methyl-N-methylthreonine (MeBmt), N-methyl-leucine (MeLeu), aminoisobutyric acid, statine, and N-methyl-alanine (MeAla). Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

In certain embodiments, angiotensin-(1-7) peptides contain one or more L-amino acids, D-amino acids, and/or un-natural amino acids.

In addition to peptides containing only naturally occurring amino acids, peptidomimetics or peptide analogs are also encompassed by the present invention. Peptide analogs are commonly used in the pharmaceutical industry as non-peptide drugs with properties analogous to those of the template peptide. The non-peptide compounds are termed “peptide mimetics” or peptidomimetics (Fauchere et al., Infect. Immun. 54:283-287 (1986); Evans et al., J. Med. Chem. 30:1229-1239 (1987)). Peptide mimetics that are structurally related to therapeutically useful peptides and may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1(3):267 (1983); Spatola et al. Life Sci. 38:1243-1249 (1986); Hudson et al. Int. J. Pept. Res. 14:177-185 (1979); and Weinstein. B., 1983, Chemistry and Biochemistry, of Amino Acids, Peptides and Proteins, Weinstein eds, Marcel Dekker, New-York,). Such peptide mimetics may have significant advantages over naturally-occurring polypeptides including more economical production, greater chemical stability, enhanced pharmacological properties (e.g., half-life, absorption, potency, efficiency, etc.), reduced antigenicity and others.

Ang-(1-7) peptides also include other types of peptide derivatives containing additional chemical moieties not normally part of the peptide, provided that the derivative retains the desired functional activity of the peptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives; and (6) derivatives conjugated to a polyethylene glycol (PEG) chain.

Ang-(1-7) peptides may be obtained by any method of peptide synthesis known to those skilled in the art, including synthetic (e.g., exclusive solid phase synthesis, partial solid phase synthesis, fragment condensation, classical solution synthesis, native-chemical ligation) and recombinant techniques. For example, the peptides or peptides derivatives can be obtained by solid phase peptide synthesis, which in brief, consist of coupling the carboxyl group of the C-terminal amino acid to a resin (e.g., benzhydrylaminc resin, chloromethylated resin, hydroxymethyl resin) and successively adding N-alpha protected amino acids. The protecting groups may be any such groups known in the art. Before each new amino acid is added to the growing chain, the protecting group of the previous amino acid added to the chain is removed. Such solid phase synthesis has been disclosed, for example, by Merrifield, J. Am. Chem. Soc. 85: 2149 (1964); Vale et al., Science 213:1394-1397 (1981), in U.S. Pat. Nos. 4,305,872 and 4,316,891, Bodonsky et al. Chem. Ind. (London), 38:1597 (1966); and Pietta and Marshall, Chem. Comm. 650 (1970) by techniques reviewed in Lubell et al. “Peptides” Science of Synthesis 21.11, Chemistry of Amides. Thieme, Stuttgart, 713-809 (2005). The coupling of amino acids to appropriate resins is also well known in the art and has been disclosed in U.S. Pat. No. 4,244,946. (Reviewed in Houver-Weyl, Methods of Organic Chemistry. Vol E22a. Synthesis of Peptides and Peptidomimetics, Murray Goodman, Editor-in-Chief, Thieme. Stuttgart. N.Y. 2002).

Unless defined otherwise, the scientific and technological terms and nomenclature used herein have the same meaning as commonly understood by a person of ordinary skill to which this invention pertains. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3^{rd} edition, Cold Spring Harbor Laboratory Press, N.Y., 2001.

During any process of the preparation of an Ang-(1-7) peptide, it may be desirable to protect sensitive reactive groups on any of the molecule concerned. This may be achieved by means of conventional protecting groups such as those described in Protective Groups In Organic Synthesis by T. W. Greene & P. G. M. Wuts, 1991, John Wiley and Sons, New-York; and Peptides: chemistry and Biology by Sewald and Jakubke, 2002, Wiley-VCH, Wheinheim p. 142. For example, alpha amino protecting groups include acyl type protecting groups (e.g., trifluoroacetyl, formyl, acetyl), aliphatic urethane protecting groups (e.g., t-butyloxycarbonyl (BOC), cyclohexyloxycarbonyl), aromatic urethane type protecting groups (e.g., fluorenyl-9-methoxy-carbonyl (Fmoc), benzyloxycarbonyl (Cbz), Cbz derivatives) and alkyl type protecting groups (e.g., triphenyl methyl, benzyl). The amino acids side chain protecting groups include benzyl (for Thr and Ser), Cbz (Tyr, Thr, Ser, Arg, Lys), methyl ethyl, cyclohexyl (Asp, His), Boc (Arg, His, Cys) etc. The protecting groups may be removed at a convenient subsequent stage using methods known in the art.

Further, Ang-(1-7) peptides may be synthesized according to the FMOC protocol in an organic phase with protective groups. Desirably, the peptides are purified with a yield of 70% with high-pressure liquid chromatography (HPLC) on a C18 chromatography column and eluted with an acetonitrile gradient of 10-60%. The molecular weight of a peptide can be verified by mass spectrometry (reviewed in Fields, G. B. “Solid-Phase Peptide Synthesis” Methods in Enzymology. Vol. 289, Academic Press, 1997).

Alternatively, Ang-(1-7) peptides may be prepared in recombinant systems using, for example, polynucleotide sequences encoding the polypeptides. It is understood that a polypeptide may contain more than one of the above-described modifications within the same polypeptide.

While peptides may be effective in eliciting a biological activity in vitro, their effectiveness in vivo might be reduced by the presence of proteases. Serum proteases have specific substrate requirements. The substrate must have both L-amino acids and peptide bonds for cleavage. Furthermore, exopeptidases, which represent the most prominent component of the protease activity in serum, usually act on the first peptide bond of the peptide and require a free N-terminus (Powell et al., Pharm. Res. 10:1268-1273 (1993)). In light of this, it is often advantageous to use modified versions of peptides. The modified peptides retain the structural characteristics of the original L-amino acid peptides that confer the desired biological activity of Ang-(1-7) but are advantageously not readily susceptible to cleavage by protease and/or exopeptidases.

Systematic substitution of one or more amino acids of a consensus sequence with D-amino acid of the same type (e.g., D-lysine in place of L-lysine) may be used to generate more stable peptides. Thus, a peptide derivative or peptidomimetic of the present invention may be all L, all D or mixed D, L peptide, in either forward or reverse order. The presence of an N-terminal or C-terminal D-amino acid increases the in vivo stability of a peptide since peptidases cannot utilize a D-amino acid as a substrate (Powell et al., Pharm. Res. 10:1268-1273 (1993)). Reverse-D peptides are peptides containing D-amino acids, arranged in a reverse sequence relative to a peptide containing L-amino acids. Thus, the C-terminal residue of an L-amino acid peptide becomes N-terminal for the D-amino acid peptide, and so forth. Reverse D-peptides retain the same secondary conformation and therefore similar activity, as the L-amino acid peptides, but are more resistant to enzymatic degradation in vitro and in vivo, and thus can have greater therapeutic efficacy than the original peptide (Brady and Dodson, Nature 368:692-693 (1994); Jameson et al., Nature 368:744-746 (1994)). Similarly, a reverse-L peptide may be generated using standard methods where the C-terminus of the parent peptide becomes takes the place of the N-terminus of the reverse-L peptide. It is contemplated that reverse L-peptides of L-amino acid peptides that do not have significant secondary structure (e.g., short peptides) retain the same spacing and conformation of the side chains of the L-amino acid peptide and therefore often have the similar activity as the original L-amino acid peptide. Moreover, a reverse peptide may contain a combination of L- and D-amino acids. The spacing between amino acids and the conformation of the side chains may be retained resulting in similar activity as the original L-amino acid peptide.

Another effective approach to confer resistance to peptidases acting on the N-terminal or C-terminal residues of a peptide is to add chemical groups at the peptide termini, such that the modified peptide is no longer a substrate for the peptidase. One such chemical modification is glycosylation of the peptides at either or both termini. Certain chemical modifications, in particular N-terminal glycosylation, have been shown to increase the stability of peptides in human scrum (Powell et al., Pharm. Res. 10:1268-1273 (1993)). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified peptides consisting of peptides bearing an N-terminal acetyl group and/or a C-terminal amide group.

Substitution of non-naturally-occurring amino acids for natural amino acids in a subsequence of the peptides can also confer resistance to proteolysis. Such a substitution can, for instance, confer resistance to proteolysis by exopeptidases acting on the N-terminus without affecting biological activity. Examples of non-naturally-occurring amino acids include α,α-disubstituted amino acids, N-alkyl amino acids, C-α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. Furthermore, the synthesis of peptides with non-naturally-occurring amino acids is routine in the art.

In addition, constrained peptides comprising a consensus sequence or a substantially identical consensus sequence variation may be generated by methods well known in the art (Rizo and Gicrasch, Ann. Rev. Biochem. 61:387-418 (1992)). For example, constrained peptides may be generated by adding cysteine residues capable of forming disulfide bridges and, thereby, resulting in a cyclic peptide. Cyclic peptides can be constructed to have no free N- or C-termini. Accordingly, they are not susceptible to proteolysis by exopeptidases, although they may be susceptible to endopeptidases, which do not cleave at peptide termini. The amino acid sequences of the peptides with N-terminal or C-terminal D-amino acids and of the cyclic peptides are usually identical to the sequences of the peptides to which they correspond, except for the presence of N-terminal or C-terminal D-amino acid residue, or their circular structure, respectively.

Cyclic Peptides

In some embodiments, a functional equivalent, analogue or derivative of naturally-occurring Ang-(1-7) is a cyclic peptide. As used herein, a cyclic peptide has an intramolecular covalent bond between two non-adjacent residues. The intramolecular bond may be a backbone to backbone, side-chain to backbone or side-chain to side-chain bond (i.e., terminal functional groups of a linear peptide and/or side-chain functional groups of a terminal or interior residue may be linked to achieve cyclization). Typical intramolecular bonds include disulfide, amide and thioether bonds. A variety of means for cyclizing polypeptides are well known in the art, as are many other modifications that can be made to such peptides. For a general discussion, see International Patent Publication Nos. WO 01/53331 and WO 98/02452, the contents of which are incorporated herein by reference. Such cyclic bonds and other modifications can also be applied to the cyclic peptides and derivative compounds of this invention.

Cyclic peptides as described herein may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. Cyclic peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Suitable derivatives include amino acids having an N-acetyl group (such that the amino group that represents the N-terminus of the linear peptide prior to cyclization is acetylated) and/or a C-terminal amide group (i.e., the carboxy terminus of the linear peptide prior to cyclization is amidated). Residues other than common amino acids that may be present with a cyclic peptide include, but are not limited to, penicillamine, β,β-tetramethylene cysteine, β,β-pentamethylene cysteine, β-mercaptopropionic acid, β,β-pentamethylene-β-mercaptopropionic acid, 2-mercaptobenzene, 2-mercaptoaniline, 2-mercaptoproline, ornithine, diaminobutyric acid, α-aminoadipic acid, m-aminomethylbenzoic acid and α,β-diaminopropionic acid.

Following synthesis of a linear peptide, with or without N-acetylation and/or C-amidation, cyclization may be achieved by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. Alternatively, strong oxidizing agents such as I₂ and K₃Fe(CN)₆ can be used to form disulfide linkages. Those of ordinary skill in the art will recognize that care must be taken not to oxidize the sensitive side chains of Met, Tyr, Tip or His. Within further embodiments, cyclization may be achieved by amide bond formation. For example, a peptide bond may be formed between terminal functional groups (i.e., the amino and carboxy termini of a linear peptide prior to cyclization). Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate. Methods for forming amide bonds are generally well known in the art. Within one such method, carbodiimide-mediated lactam formation can be accomplished by reaction of the carboxylic acid with DCC, DIC, ED AC or DCCI, resulting in the formation of an O-acylurea that can be reacted immediately with the free amino group to complete the cyclization. Alternatively, cyclization can be performed using the azide method, in which a reactive azide intermediate is generated from an alkyl ester via a hydrazide. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF.

Exemplary Angiotensin-(1-7) Peptides

Linear Angiotensin(1-7) Peptides

In certain aspects, the invention provides linear angiotensin-(1-7) peptides. As discussed above, the structure of naturally-occurring Ang-(1-7) is as follows:

(SEQ ID NO: 1)
Asp¹-Arg²-Val³-Tyr⁴-Ile⁵-His⁶-Pro⁷

The peptides and peptide analogs of the invention can be generally represented by the following sequence:

(SEQ ID NO: 4)
Xaa¹-Xaa²-Xaa³-Xaa⁴-Xaa⁵-Xaa⁶-Xaa⁷,

or a pharmaceutically acceptable salt thereof.

Xaa¹ is any amino acid or a dicarboxylic acid. In certain embodiments, Xaa¹ is Asp, Glu, Asn, Acpc (1-aminocyclopentane carboxylic acid), Ala, Me₂Gly (N,N-dimethylglycine), Pro, Bet (betaine, 1-carboxy-N,N,N-trimethylmethanaminium hydroxide), Glu, Gly, Asp, Sar (sarcosine) or Suc (succinic acid). In certain such embodiments, Xaa¹ is a negatively-charged amino acid, such as Asp or Glu, typically Asp.

Xaa² is Arg, Lys, Ala, Cit (citrulline), Orn (ornithine), acetylated Ser, Sar, D-Arg and D-Lys. In certain embodiments, Xaa² is a positively-charged amino acid such as Arg or Lys, typically Arg.

Xaa³ is Val, Ala, Leu, Nle (norleucine), Ile, Gly, Lys, Pro, HydroxyPro (hydroxyproline), Aib (2-aminoisobutyric acid), Acpc or Tyr. In certain embodiments, Xaa³ is an aliphatic amino acid such as Val, Lcu, Ile or Nlc, typically Val or Nlc.

Xaa⁴ is Tyr, Tyr(PO₃), Thr, Ser, homoSer (homoserine), azaTyr (aza-α¹-homo-L-tyrosine) or Ala. In certain embodiments, Xaa⁴ is a hydroxyl-substituted amino acid such as Tyr, Ser or Thr, typically Tyr.

Xaa⁵ is Ile, Ala, Leu, norLeu, Val or Gly. In certain embodiments, Xaa⁵ is an aliphatic amino acid such as Val, Leu, Ile or Nle, typically Ile.

Xaa⁶ is His, Arg or 6-NH₂-Phe (6-aminophenylalanine). In certain embodiments, Xaa⁶ is a fully or partially positively-charged amino acid such as Arg or His.

Xaa⁷ is Cys, Pro or Ala.

In certain embodiments, one or more of Xaa¹-Xaa⁷ is identical to the corresponding amino acid in naturally-occurring Ang-(1-7). In certain such embodiments, all but one or two of Xaa¹-Xaa⁷ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Xaa³ is Nle. When Xaa³ is Nle, one or more of Xaa¹-Xaa² and Xaa⁴⁻⁷ are optionally identical to the corresponding amino acid in naturally-occurring Ang-(1-7). In certain such embodiments, all but one or two of Xaa¹-Xaa² and Xaa⁴⁻⁷ are identical to the corresponding amino acid in naturally-occurring Ang-(1-7). In other embodiments, all of Xaa¹-Xaa² and Xaa⁴⁻⁷ are identical to the corresponding amino acid in naturally-occurring Ang-(1-7), resulting in the amino acid sequence: Asp¹-Arg²-Nle³-Tyr⁴-Ile⁵-His⁶-Pro⁷ (SEQ ID NO: 5).

In certain embodiments, the peptide has the amino acid sequence Asp¹-Arg²-Val³-Ser⁴-Ile⁵-His⁶-Cys⁷ (SEQ ID NO: 2) or Asp¹-Arg²-Val³-ser⁴-Ile⁵-His⁶-Cys⁷ (SEQ ID NO: 6).

Exemplary Cyclic Angiotensin (1-7) Peptides

In certain aspects, the invention provides a cyclic angiotensin-(1-7) (Ang-(1-7)) peptide analog comprising a linkage, such as between the side chains of amino acids corresponding to positions Tyr⁴ and Pro⁷ in Ang. These peptide analogs typically comprise 7 amino acid residues, but can also include a cleavable sequence. As discussed in greater detail below, the invention includes fragments and analogs where one or more amino acids are substituted by another amino acid (including fragments), for example, Asp¹-Arg²-Val³-Ser⁴-Ile⁵-His⁶-Cys⁷ (SEQ ID NO: 22), wherein a linkage is formed between Ser⁴ and Cys⁷.

Although the following section describes aspects of the invention in terms of a thioether bond linking residues at the 4- and 7-positions, it should be understood that other linkages (as described above) could replace the thioether bridge and that other residues could be cyclized. A thioether bridge is also referred to as a monosulfide bridge or, in the case of Ala-S-Ala, as a lanthionine bridge. Thioether bridge-containing peptides can be formed by two amino acids having one of the following formulas:

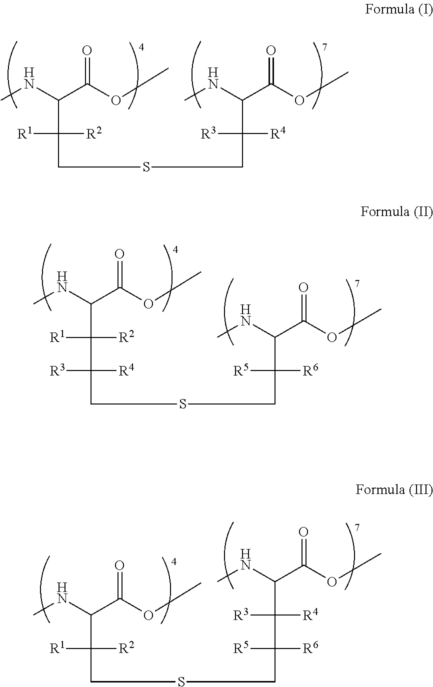

In these formulae, R¹, R², R³, R⁴, R⁵ and R⁶ are independently —H, an alkyl (e.g., C₁-C₆ alkyl, C₁-C₄ alkyl) or an aralkyl group, where the alkyl and aralkyl groups are optionally substituted with one or more halogen, —OH or —NRR′ groups (where R and R′ are independently —H or C₁-C₄ alkyl). In certain embodiments, R′, R², R³, R⁴, R⁵ and R⁶ are each independently —H or —CH₃, such where all are —H.

In certain embodiments, the invention provides an Ang analog or derivative comprising a thioether bridge according to formula (I). Typically, R¹, R², R³ and R⁴ are independently selected from —H and —CH₃. Peptides comprising a thioether bridge according to formula (I) can be produced, for example, by lantibiotic enzymes or by sulfur extrusion of a disulfide. In one example, the disulfide from which the sulfur is extruded can be formed by D-cysteine in position 4 and L-cysteine in position 7 or by D-cysteine in position 4 and L-penicillamine in position 7 (see, e.g., Galande, Trent and Spatola (2003) Biopolymers 71, 534-551).

In other embodiments, the linkage of the two amino acids can be the bridges depicted in Formula (II) or Formula (III). Peptides comprising a thioether bridge according to Formula (II) can be made, for example, by sulfur extrusion of a disulfide formed by D-homocysteinc in position 4 and L-cysteine in position 7. Similarly, peptides comprising a thioether bridge as in Formula (III) can be made, for example, by sulfur extrusion of a disulfide formed by D-cysteine in position 4 and L-homocysteine in position 7.

As discussed above, the Ang analogs and derivatives of the invention vary in length and amino acid composition. The Ang analogs and derivatives of the invention preferably have biological activity or are an inactive precursor molecule that can be proteolytically activated (such as how angiotensin(I), with 10 amino acids, is converted to active fragments by cleavage of 2 amino acids). The size of an Ang analog or derivative can vary but is typically between from about 5 to 10 amino acids, as long as the “core” pentameric segment comprising the 3-7 Nle-thioether-ring structure is encompassed. The amino acid sequence of an analog or derivative of the invention can vary, typically provided that it is biologically active or can become proteolytically activated. Biological activity of an analog or derivative can be determined using methods known in the art, including radioligand binding studies, in vitro cell activation assays and in vivo experiments. See, for example, Godeny and Sayeski, (2006) Am. J. Physiol. Cell. Physiol. 291:C1297-1307; Sarr et al., Cardiovasc. Res. (2006) 71:794-802; and Koziarz et al., (1933) Gen. Pharmacol. 24:705-713.

Ang analogs and derivatives where only the length of the peptide is varied include the following:

a 4,7-cyclized analog designated [Cyc⁴⁻⁷]Ang-(1-7), which is derived from natural Ang-(1-7) (Asp¹-Arg²-Val³-Cyc⁴-Ile⁵-His⁶-Cyc⁷, SEQ ID NO:7).

a 4,7-cyclized analog designated [Nle³, Cyc⁴⁻⁷]Ang-(1-10), which is derived from natural Angiotensin I (Ang-(1-10)) (Asp¹-Arg²-Nle³-Cyc⁴-Ile⁵-His⁶-Cyc⁷-Phe⁸-His⁹-Leu¹⁰, SEQ ID NO:8);

a 4,7-cyclized analog designated [Nle³, Cyc⁴⁻⁷]Ang-(1-8), which is derived from natural Angiotensin II (Ang-(1-8)) (Asp′-Arg²-Nle³-Cyc⁴-Ile⁵-His⁶-Cyc⁷-Phe⁸, SEQ ID NO:9);

a 4,7-cyclised analog designated [Nle³, Cyc⁴⁻⁷]Ang-(2-8), which is derived from natural Angiotensin III (Ang-(2-8)) (Arg²-Nle³-Cyc⁴-Ile⁵-His⁶-Cyc⁷-Phe⁸, SEQ ID NO:10);

a 4,7-cyclised analog designated [Nle³, Cyc⁴⁻⁷]Ang-(3-8), which is derived from natural Angiotensin IV (Ang-(3-8)) (Nle³-Cyc⁴-Ile⁵-His⁶-Cyc⁷-Phe⁸, SEQ ID NO:11);

a 4,7-cyclised analog designated [Nle³, Cyc⁴⁷]Ang-(1-7) derived from natural Ang-(1-7) (Asp′-Arg²-Nle³-Cyc⁴-Ile⁵-His⁶-Cyc⁷, SEQ ID NO:12); and

a 4,7-cyclised analog designated [Nle³, Cyc⁴⁻⁷]Ang-(1-9) derived from natural Ang-(1-9) (Asp′-Arg²-Nle³-Cyc⁴-Ile⁵-His⁶-Cyc⁷-Phe⁸-His⁹, SEQ ID NO:13).

These analogs can have one of the thioether bridges shown in Formulae (I)-(III) as the Cyc⁴⁻⁷ moiety, for example, where Cyc⁴ and Cyc⁷ are represented by Formula (I), such as where R¹-R⁴ are each —H or —CH₃, typically —H.

As compared to the amino acid sequence of the natural angiotensin peptide, the amino acids at positions 4 and 7 of the Cyc⁴⁻⁷ analog are modified to allow introduction of the thioether-ring structures shown above. In addition to the length of the Ang analogs, the amino acids at positions other than 3, 4 and 7 can be the same or different from the naturally-occurring peptide, typically provided that the analog retains a biological function. One example is Asp¹-Arg²-Val³-Ser⁴-Ile⁵-His⁶-Cys⁷ (SEQ ID NO:22). For analogs of inactive precursors, like [Cyc⁴⁻⁷]Ang-(1-10), biological function refers to one or both of an analog's susceptibility to angiotensin-converting enzymes that can cleave it to a biologically active fragment (e.g. Ang-(1-8) or Ang-(1-7)) or the biological activity of the fragment itself. In certain embodiments, an Ang analog or derivative of the invention has no intrinsic function but inhibits the effects of one or more naturally-occurring angiotensin compounds.

In certain embodiments, an Ang analog of the invention is represented by Formula (IV):

(IV, SEQ ID NO: 14)
Xaa¹-Xaa²-Xaa³-Cyc⁴-Xaa⁵-Xaa⁶-Cyc⁷

Xaa¹ is any amino acid, but typically a negatively-charged amino acid such as Glu or Asp, more typically Asp.

Xaa² is a positively-charged amino acid such as Arg or Lys, typically Arg.

Xaa³ is an aliphatic amino acid, such as Leu, Ile or Val, typically Val.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II) and (III). Typically, the R groups in Formulas (I), (II) and (III) are —H or —CH₃, especially —H.

In certain embodiments, one or more of Xaa¹-Xaa⁶ (excluding Cyc⁴ and Cyc⁷) is identical to the corresponding amino acid in naturally-occurring Ang-(1-7). In certain such embodiments, all but one or two of Xaa¹-Xaa⁶ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Cyc⁴ and Cyc⁷ are independently selected from Abu (2-aminobutyric acid) and Ala (alanine), where Ala is present in at least one position. Thus, cyclic analogs can have a thioether linkage formed by -Ala⁴-S-Ala⁷- (Formula (T), where R′—R⁴ are each —H); -Ala⁴-S-Abu⁷- (Formula (I): R¹-R³ are —H and R⁴ is —CH₃) or -Abu⁴-S-Ala⁷- (Formula (I): R¹, R³ and R⁴ are —H and R² is —CH₃). Specific examples of cyclic analogs comprise a -Abu⁴-S-Ala⁷- or -Ala⁴-S-Ala⁷-linkage.

In certain embodiments, the invention provides an Ang-(1-7) analog with a thioether-bridge between position 4 and position 7 having the amino acid sequence Asp¹-Arg²-Val³-Abu⁴-Ile⁵-His⁶-Ala⁷ (SEQ ID NO:15) or the amino acid sequence Asp¹-Arg²-Val³-Ala⁴-Ile⁵-His⁶-Ala⁷ (SEQ ID NO:16), which are represented by the following structural diagrams:

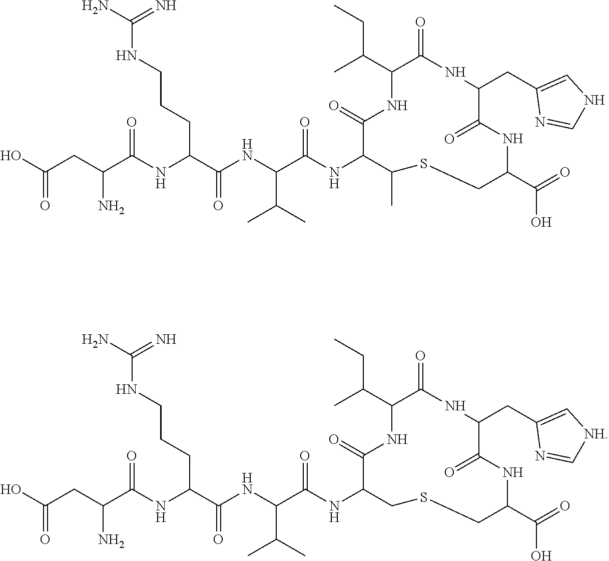 [SMILES: CCC(C)C1NC(=O)C(NC(=O)C(NC(=O)C(CCCNC(=N)N)NC(=O)C(N)CC(=O)O)C(C)C)C(C)SCC(C(=O)O)NC(=O)C(Cc2c[nH]cn2)NC1=O ; CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In certain embodiments, an Ang analog or derivative of the invention is represented by Formula (V):

(V, SEQ ID NO: 17)
Xaa¹-Xaa²-Nle³-Cyc⁴-Xaa⁵-Xaa⁶-Cyc⁷-Xaa⁸-Xaa⁹-Xaa¹⁰

As discussed above, one or more of Xaa¹, Xaa², Xaa⁸, Xaa⁹ and Xaa¹⁰ are absent in certain embodiments. For example, (1) Xaa¹⁰ is absent, (2) Xaa⁹ and Xaa¹⁰ are absent, (3) Xaa⁸, Xaa⁹ and Xaa¹⁰ are absent, (4) Xaa¹ is absent, (5) Xaa¹ and Xaa¹⁰ are absent, (6) Xaa¹, Xaa⁹ and Xaa¹⁰ are absent, (7) Xaa¹, Xaa⁸, Xaa⁹ and Xaa¹⁰ are absent, (8) Xaa¹ and Xaa² are absent, (9) Xaa¹, Xaa² and Xaa¹⁰ are absent, (10) Xaa¹, Xaa², Xaa⁹ and Xaa¹⁰ are absent, or (11) Xaa¹, Xaa², Xaa⁸, Xaa⁹ and Xaa¹⁰ are absent. For each of these embodiments, the remaining amino acids have the values described below.

Xaa¹, when present, is any amino acid, but typically a negatively charged amino acid such as Glu or Asp, more typically Asp.

Xaa², when present, is a positively charged amino acid such as Arg or Lys, typically Arg.

Nle³ is norleucine.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Nle, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II) and (III). Typically, the R groups in Formulae (I), (II) and (III) are —H or —CH₃, especially —H.

Xaa⁸, when present, is an amino acid other than Pro, typically Phe or Ile. In certain embodiments, Ile results in an inhibitor of Ang(1-8). In certain embodiments, Phe maintains the biological activity of Ang(1-8) or Ang(1-10).

Xaa⁹, when present, is His.

Xaa¹⁰, when present, is an aliphatic residue, for example, Ile, Val or Leu, typically Leu.

In certain embodiments, one or more of Xaa¹-Xaa¹⁰ (excluding Nle³, Cyc⁴ and Cyc⁷) is identical to the corresponding amino acid in naturally-occurring Ang (including Ang-(1-7), Ang(1-8), Ang(1-9), Ang(1-10), Ang(2-7), Ang(2-8), Ang(2-9), Ang(2-10), Ang(3-8), Ang(3-9) and Ang(3-10). In certain such embodiments, all but one or two of Xaa¹-Xaa¹⁰ (for those present) are identical to the corresponding amino acid in naturally-occurring Ang. In other embodiments, all of Xaa¹-Xaa¹⁰ (for those present) are identical to the corresponding amino acid in naturally-occurring Ang.

In certain embodiments, Cyc⁴ and Cyc⁷ are independently selected from Abu (2-aminobutyric acid) and Ala (alanine), where Ala is present at at least one position. Thus, encompassed are cyclic analogs comprising a thioether linkage formed by -Ala⁴-S-Ala⁷-(Formula (I), where R¹-R⁴ are each —H); -Ala⁴-S-Abu⁷- (Formula (I): R¹-R³ are —H and R⁴ is —CH₃) or -Abu⁴-S-Ala⁷- (Formula (I): R¹, R³ and R⁴ are —H and R² is —CH₃). Specific cyclic analogs comprise a -Abu⁴-S-Ala⁷- or -Ala⁴-S-Ala⁷- linkage.

In particular, the invention provides an Ang-(1-7) analog or derivative with a thioether-bridge between position 4 and position 7 having the amino acid sequence Asp¹-Arg²-Nle³-Abu⁴-Ile⁵-His⁶-Ala⁷ (SEQ ID NO:18) or the amino acid sequence Asp¹-Arg²-Nle³-Ala⁴-Ile⁵-His⁶-Ala⁷ (SEQ ID NO:19).

In another aspect, the invention provides an Ang-(1-8) analog or derivative with a thioether-bridge between position 4 and position 7 having Ang-(1-8) antagonistic activity, in particular an Ang(1-8) analog or derivative having the amino acid sequence Asp¹-Arg²-Nle³-Abu⁴-Ile⁵-His⁶-Ala⁷-Ile⁸ (SEQ ID NO:20) or the amino acid sequence Asp¹-Arg²-Nle³-Ala⁴-Ile⁵-His⁶-Ala⁷-Ile⁸ (SEQ ID NO:21).

Ang (1-7) Receptor Agonists

In some embodiments, the present invention provides methods of treating muscular dystrophy including administering to a subject who is suffering from or susceptible to muscular dystrophy an angiotensin (1-7) receptor agonist. As used herein, the term “angiotensin-(1-7) receptor agonist” encompasses any molecule that has a positive impact in a function of an angiotensin-(1-7) receptor, in particular, the G-protein coupled Mas receptor. In some embodiments, an angiotensin-(1-7) receptor agonist directly or indirectly enhances, strengthens, activates and/or increases an angiotensin-(1-7) receptor (i.e., the Mas receptor) activity. In some embodiments, an angiotensin-(1-7) receptor agonist directly interacts with an angiotensin-(1-7) receptor (i.e., the Mas receptor). Such agonists can be peptidic or non-peptidic including, e.g., proteins, chemical compounds, small molecules, nucleic acids, antibodies, drugs, ligands, or other agents. In some embodiments, the angiotensin (1-7) receptor agonist is a non-peptidic agonist.

An exemplary class of angiotensin-(1-7) receptor agonists are 1-(p-thienylbenzyl)imidazoles. Examples of these non-peptide angiotensin-(1-7) receptor agonists are represented by Structural Formula (VI):

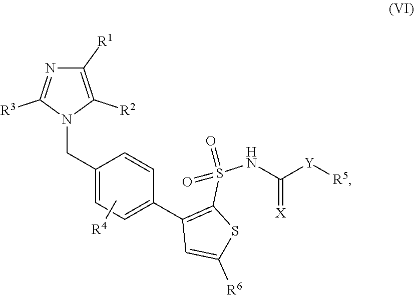

or pharmaceutically acceptable salts thereof, wherein:

R¹ is halogen, hydroxyl, (C₁-C₄)-alkoxy, (C₁-C₈)-alkoxy wherein 1 to 6 carbon atoms are replaced by the heteroatoms O, S, or NH (preferably by O), (C₁-C₄)-alkoxy substituted by a saturated cyclic ether such as tetrahydropyran or tetrahydrofuran, O—(C₁-C₄)-alkenyl, O—(C₁-C₄)-alkylaryl, or aryloxy that is unsubstituted or substituted by a substituent selected from halogen, (C₁-C₃)-alkyl, (C₁-C₃)-alkoxy and trifluoromethyl;

R² is CHO, COOH, or (3) CO—O—(C₁-C₄)-alkyl;

R³ is (C₁-C₄)-alkyl or aryl;

R⁴ is hydrogen, halogen (chloro, bromo, fluoro), or (C₁-C₄)-alkyl;

X is oxygen or sulfur;

Y is oxygen or —NH—;

R⁵ is hydrogen, (C₁-C₆)-alkyl; or (C₁-C₄)-alkylaryl, where R⁵ is hydrogen when Y is —NH—; and

R⁶ is (C₁-C₅)-alkyl.

In certain embodiments, R¹ is not halogen when R² is COOH or CO—O—(C₁-C₄)-alkyl.

In some embodiments, an angiotensin-(1-7) receptor agonist is AVE 0991, 5-formyl-4-methoxy-2-phenyl-1[[4-[2-(ethylaminocarbonylsulfonamido)-5-isobutyl-3-thienyl]-phenyl]-methyl]-imidazole, which is represented by the following structure:

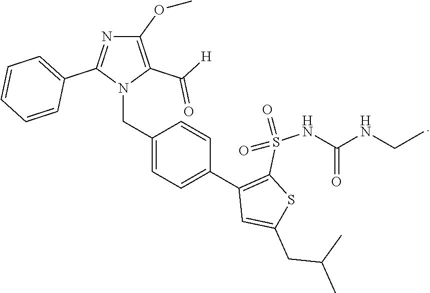 [SMILES: CCNC(=O)NS(=O)(=O)c1sc(CC(C)C)cc1-c1ccc(Cn2c(-c3ccccc3)nc(OC)c2C=O)cc1]

Another exemplary class of angiotensin-(1-7) receptor agonists are p-thienylbenzylamides. Examples of these non-peptide angiotensin-(1-7) receptor agonists are represented by Structural Formula (VII):

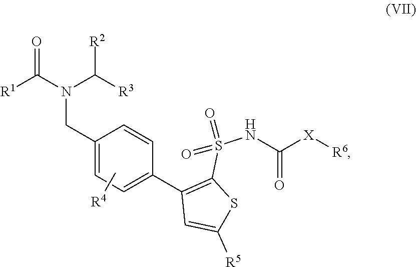

or a pharmaceutically acceptable salt thereof, wherein:

R¹ is (C₁-C₅)-alkyl that is unsubstituted or substituted by a radical chosen from NH₂, halogen, O—(C₁-C₃)-alkyl, CO—O—(C₁-C₃)-alkyl and CO₂H, (C₃-C₈)-cycloalkyl, (C₁-C₃)-alkyl-(C₃-C₈)-cycloalkyl, (C₆-C₁₀)-aryl that is unsubstituted or substituted by a radical chosen from halogen and O—(C₁-C₃)-alkyl, (C₁-C₃)-alkyl-(C₆-C₁₀)-aryl where the aryl radical is unsubstituted or substituted by a radical chosen from halogen and O—(C₁-C₃)-alkyl, (C₁-C₅)-heteroaryl, or (C₁-C₃)-alkyl-(C₁-C₅)-heteroaryl;

R² is hydrogen, (C₁-C₆)-alkyl that is unsubstituted or substituted by a radical chosen from halogen and O—(C₁-C₃)-alkyl, (C₃-C₈)-cycloalkyl, (C₁-C₃)-alkyl-(C₃-C₈)-cycloalkyl, (C₆-C₁₀)-aryl that is unsubstituted or substituted by a radical chosen from among halogen, O—(C₁-C₃)-alkyl and CO—O—(C₁-C₃)-alkyl, or (C₁-C₃)-alkyl-(C₆-C₁₀)-aryl that is unsubstituted or substituted by a radical chosen from halogen and O—(C₁-C₃)-alkyl;

R³ is hydrogen, COOH, or COO—(C₁-C₄)-alkyl;

R⁴ is hydrogen, halogen; or (C₁-C₄)-alkyl;

R⁵ is hydrogen or (C₁-C₆)-alkyl;

R⁶ is hydrogen, (C₁-C₆)-alkyl, (C₁-C₃)-alkyl-(C₃-C₈)-cycloalkyl, or (C₂-C₆)-alkenyl; and

X is oxygen or NH.

Additional examples of angiotensin-(1-7) receptor agonists are described in U.S. Pat. No. 6,235,766, the contents of which are incorporated by reference herein.

Various angiotensin-(1-7) receptor agonists described above can be present as pharmaceutically acceptable salts. As used herein, “a pharmaceutically acceptable salt” refers to salts that retain the desired activity of the peptide or equivalent compound, but preferably do not detrimentally affect the activity of the peptide or other component of a system, which uses the peptide. Examples of such salts are acid addition salts formed with inorganic acids, for example, hydrochloric acid, hydrobromic acid, sulfuric acid, phosphoric acid, nitric acid, and the like. Salts may also be formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, and the like. Salts formed from a cationic material may utilize the conjugate base of these inorganic and organic acids. Salts may also be formed with polyvalent metal cations such as zinc, calcium, bismuth, barium, magnesium, aluminum, copper, cobalt, nickel and the like or with an organic cation formed from N,N′-dibenzylethylenediamine or ethylenediamine, or combinations thereof (e.g., a zinc tannate salt). The non-toxic, physiologically acceptable salts are preferred.

The salts can be formed by conventional means such as by reacting the free acid or free base forms of the product with one or more equivalents of the appropriate acid or base in a solvent or medium in which the salt is insoluble, or in a solvent such as water which is then removed in vacuo or by freeze-drying, or by exchanging the cations of an existing salt for another cation on a suitable ion exchange resin.

An alkyl group is a straight chained or branched non-aromatic hydrocarbon that is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained or branched non-aromatic hydrocarbon that is includes one or more double bonds. Typically, a straight chained or branched alkenyl group has from 2 to about 20 carbon atoms, preferably from 2 to about 10. Examples of straight chained and branched alkenyl groups include ethenyl, n-propenyl, and n-butenyl.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

An aralkyl group is an alkyl group substituted by an aryl group. Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Pharmaceutical Compositions

In accordance with the methods of the invention, an angiotensin (1-7) peptide or angiotensin (1-7) receptor agonist as described herein can be administered to a subject alone (e.g., as a purified peptide or compound), or as a component of a composition or medicament (e.g., in the manufacture of a medicament for the treatment of the disease), as described herein. The compositions can be formulated with a physiologically acceptable carrier or excipient to prepare a pharmaceutical composition. The carrier and composition can be sterile. The formulation should suit the mode of administration. Methods of formulating compositions are known in the art (see, e.g., Remington's Pharmaceuticals Sciences, 17^{th} Edition, Mack Publishing Co., (Alfonso R. Gennaro, editor) (1989)).

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In a preferred embodiment, a water-soluble carrier suitable for intravenous administration is used.

The composition or medicament, if desired, can also contain minor amounts of wetting or emulsifying agents, or pH buffering agents, some of which are further discussed below. The composition can be a liquid solution, suspension, emulsion, tablet, pill, capsule, sustained release formulation, or powder. The composition can also be formulated as a suppository, with traditional binders and carriers such as triglycerides. Oral formulations can include standard carriers such as pharmaceutical grades of mannitol, lactose, starch, magnesium stearate, polyvinyl pyrrolidone, sodium saccharine, cellulose, magnesium carbonate, etc.

The composition or medicament can be formulated in accordance with the routine procedures as a pharmaceutical composition adapted for administration to human beings. For example, in a preferred embodiment, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

In some embodiments, provided compositions, including those provided as pharmaceutical formulations, comprise a liquid carrier such as but not limited to water, saline, phosphate buffered saline, Ringer's solution, dextrose solution, serum-containing solutions, Hank's solution, other aqueous physiologically balanced solutions, oils, esters and glycols.

An angiotensin (1-7) peptide or angiotensin (1-7) receptor agonist as described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Oral Formulations

In some embodiments, a suitable pharmaceutical composition is an oral formulation. It is contemplated that any medically-acceptable oral formulation may be used within the scope of the present invention.

In some embodiments, provided compositions include at least one pH-lowering agent. It is contemplated that a pH-lowering agent suitable for use in some embodiments of the present invention include any pharmaceutically acceptable pH-lowering agent, or combination of pH-lowering agents, that are a) not toxic to the gastrointestinal tract, b) are capable of either delivering hydrogen ions or capable of inducing higher hydrogen ion content from the local environment, and/or c) that are capable of being orally administered in an amount sufficient to lower the local intestinal pH below the pH optima for proteases found there. Various tests may be used to determine if a pH-lowering agent is suitable for the present invention and what amount is appropriate. For example, a pH-lowering agent or combination of pH-lowering agents is suitable for the present invention if a particular amount, when added to a solution of 10 milliliters of 0.1M sodium bicarbonate lowers the pH of the solution to no higher than 5.5, 4.7, or 3.5. In some embodiments, an amount of pH-lowering agent or agents may be added to lower pH, in a solution of 10 milliliters of 0.1M sodium bicarbonate, to no higher than 3.4, 3.2, 3.0, or 2.8.

In some embodiments, a suitable pH-lowering agent or agents include at least one pH-lowering agent that has a pKa no higher than 4.2 (e.g., no higher than 4.0, 3.8, 3.6, 3.4, 3.2, 3.0 or 2.8). Exemplary pH-lowering agents suitable for the present invention include, but are not limited to, carboxylic acids such as acetylsalicylic, acetic, ascorbic, citric, fumaric, glucuronic, glutaric, glyceric, glycocolic, glyoxylic, isocitric, isovaleric, lactic, maleic, oxaloacetic, oxalosuccinic, propionic, pyruvic, succinic, tartaric, and valeric; aluminum chloride; zinc chloride; acid salts of amino acids (or derivatives thereof) including acid salts of acetylglutamic acid, alanine, arginine, asparagine, aspartic acid, betaine, carnitine, carnosine, citrulline, creatine, glutamic acid, glycine, histidine, hydroxylysine, hydroxyproline, hypotaurine, isoleucine, leucine, lysine, methylhistidine, norleucine, ornithine, phenylalanine, proline, sarcosine, serine, taurine, threonine, tryptophan, tyrosine, and valine; certain phosphate esters including fructose 1,6 diphosphate and glucose 1,6 diphosphate may also be appropriate pH-lowering agents in certain embodiments. In particular embodiments, citric acid or tartaric acid is used as pH-lowering agent.

The quantity required of any particular pH-lowering agent or combination of pH-lowering agents may vary. Typically, a suitable amount may be determined using various tests known in the art and described herein (for example, using pH-lowering test in a solution of 10 milliliters of 0.1M sodium bicarbonate described above). As non-limiting examples, suitable amount of a pH lowering agent used in a formulation according to the present invention may be an amount of or greater than about 100 mg, 200 mg, 250 mg, 300 mg, 350 mg, 400 mg, 425 mg, 450 mg, 475 mg, 500 mg, 525 mg, 550 mg, 575 mg, 600 mg, 625 mg, 650 mg, 675, mg, 700 mg, 725 mg, 750 mg, 775 mg, 800 mg, 825 mg, 850 mg, 875 mg, 900 mg, 925 mg, 950 mg, 975 mg, or 1,000 mg. In other embodiments, the amount of citric acid used may exceed 1,000 mg.

In some embodiments, a suitable amount of a pH lowering agent (e.g., citric acid or tartaric acid) used may be measured as a percent of the total weight of a particular dosage form. As non-limiting examples, a suitable amount of a pH lowering agent used may be an amount of or greater than about 10% (e.g., of or greater than 15%, 20%, 25%, 30%, 35%, 40%, 45%, 50%, 55%, 60%, 65%, 70%, 75%, 80%, 85%, 90%, or 95%) of the total weight of a solid dosage form.

In various embodiments, a composition of the invention includes one or more absorption enhancers. As used herein, an absorption enhancer refers to an agent that increase the solubility of other components in either the aqueous or lipophilic environment into which they are released and/or enhance the uptake of an active peptide (e.g., an angiotensin (1-7) peptide) across the intestinal wall. In some embodiments, an absorption enhancer is referred to as a solubility enhancer and/or an uptake enhancer.

In some embodiments, it is possible to have a mixture of absorption enhancers wherein some provide enhanced solubility, some provide enhanced uptake, and some provide both. It is possible to have various numbers of absorption enhancers in a given embodiment including, without limitation, one, two, three, four, five, six, seven, eight, nine, or ten absorption enhancers.

Surface active agents are an example of useful absorption enhancers with properties of both solubility enhancers and uptake enhancers. In some embodiment, when surface active agents are used as absorption enhancers, they may be free flowing powders for facilitating the mixing and loading of capsules during the manufacturing process. In other embodiments when a surface active agent is used to increase the bioavailability of an angiotensin (1-7) peptide, the surface active agent may be selected from the group consisting of (a) anionic surface active agents such as cholesterol derivatives (e.g. bile acids), (b) cationic surface agents (e.g. acyl camitines, phospholipids and the like), (c) non-ionic surface active agents, and (d) mixtures of anionic surface active agents and negative charge neutralizers, and combinations thereof. Negative charge neutralizers include but are not limited to acyl carnitines, cetyl pyridinum chloride, and the like.

In some embodiments, an acid soluble bile acid and a cationic surface active agent with be used together as absorption enhancers. Acyl camitines (such as lauroyl carnitine), phospholipids and bile acids may be particularly effective absorption enhancers in some embodiments.

While a variety of absorption enhancers are suitable for use in various embodiments, the following exemplary list is intended to illustrate some embodiments of the present invention. Without limitation, some suitable absorption enhancers include: (a) salicylates such as sodium salicylate, 3-methoxysalicylate, 5-methoxysalicylate and homovanilate; (b) bile acids such as taurocholic, tauorodeoxycholic, deoxycholic, cholic, glycholic, lithocholate, chenodeoxycholic, ursodeoxycholic, ursocholic, dehydrocholic, fusidic, etc.; (c) non-ionic surfactants such as polyoxyethylene ethers (e.g. Brij 36T, Brij 52, Brij 56, Brij 76, Brij 96, Texaphor A6, Texaphor A14, Texaphor A60 etc.), p-t-octyl phenol polyoxyethylenes (Triton X-45, Triton X-100, Triton X-114, Triton X-305 etc.) nonylphenoxypoloxyethylenes (e.g. Igepal CO series), polyoxyethylene sorbitan esters (e.g. Tween-20, Tween-80 etc.); (d) anionic surfactants such as dioctyl sodium sulfosuccinate; (e) lyso-phospholipids such as lysolecithin and lysophosphatidylethanolamine; (f) acylcarnitines, acylcholines and acyl amino acids such as lauroylcarnitine, myristoylcarnitine, palmitoylcarnitine, lauroylcholine, myristoylcholine, palmitoylcholine, hexadecyllysine, N-acylphenylalanine, N-acylglycine etc.; g) water soluble phospholipids such as diheptanoylphosphatidylcholine, dioctylphosphatidylcholine etc.; (h) medium-chain glycerides which are mixtures of mono-, di- and triglycerides containing medium-chain-length fatty acids (caprylic, capric and lauric acids); (i) ethylene-diaminetetraacetic acid; (j) cationic surfactants such as cetylpyridinium chloride; (k) fatty acid derivatives of polyethylene glycol such as Labrasol, Labrafac, etc.; and (l) alkylsaccharides such as lauroyl maltoside, lauroyl sucrose, myristoyl sucrose, palmitoyl sucrose, etc.

In some embodiments, the absorption enhancer(s) will be present in a quantity measured as a percent by weight, relative to the overall weight of the pharmaceutical composition (typically exclusive of enteric coating). By way of additional non-limiting example, the quantity of absorption enhancer present in an embodiment may range from 0.1 to 20 percent by weight; from 0.5 to 20 percent by weight; from 1.0 to 20 percent by weight, from 2.0 to 20 percent by weight, from 3.0 to 20 percent by weight, from 4.0 to 20 percent by weight, from from 5.0 to 20 percent by weight, from 5.0 to 15 percent by weight, from 5.0 to 14 percent by weight, from 5.0 to 13 percent by weight, from 5.0 to 12 percent by weight, from 5.0 to 12 percent by weight, from 5.0 to 11 percent by weight, from 5.0 to 10 percent by weight, from 6.0 to 10 percent by weight, from 7.0 to 10 percent by weight, from 8.0 to 10 percent by weight, from 9.0 to 10 percent by weight, from 5.0 to 9.0 percent by weight, from 5.0 to 8.0 percent by weight, from 5.0 to 7.0 percent by weight, and from 5.0 to 6.0 percent by weight.

In some embodiments, the weight ratio of pH-lowering agent(s) to absorption enhancer(s) may be about 3:1, 4:1, 5:1, 6:1, 7:1, 8:1, 9:1, 10:1, 11:1, 12:1, 13:1, 14:1, 15:1, 16:1, 17:1, 18:1, 19:1, 20:1 or between any two of the foregoing exemplary ratios. The total weight of all pH-lowering agents and the total weight of all absorption enhancers in a given pharmaceutical composition is included in the foregoing exemplary ratios. For example, if a pharmaceutical composition includes two pH-lowering agents and three absorption enhancers, the foregoing ratios will be computed on the total combined weight of both pH-lowering agents and the total combined weight of all three absorption enhancers.

In some embodiments, the absorption enhancer(s) will be soluble at acid pH, such as less than pH 5.5, and in particular, between pH 3.0 and pH 5.0.

In some embodiments, provided compositions comprise one or more protective vehicles. As used herein, a protective vehicle refers to any protective component and/or structure, such as a carrier, a layer, a coating or other vehicle, that protects an active peptide (e.g., an angiotensin (1-7) peptide) from stomach proteases. Typically, a protective vehicle dissolves eventually so that the active and other ingredients in a particular dosage form may be released. A common form of protective vehicle is an enteric coating. In some embodiments, a suitable enteric costing may prevent breakdown of the pharmaceutical composition of the invention in 0.1N HCl for at least two hours, then capable of permitting complete release of all contents of the pharmaceutical composition within thirty minutes after pH is increased to 6.3 in a dissolution bath in which said composition is rotating at 100 revolutions per minute.

Many enteric coatings are known in the art and are useful in one or more embodiments. Non-limiting examples of enteric coatings include cellulose acetate phthalate, hydroxypropyl methylethylcellulose succinate, hydroxypropyl methylcellulose phthalate, carboxyl methylethylcellulose and methacrylic acid-methyl methacrylate copolymer. In some embodiments, an angiotensin (1-7) peptide, absorption enhancers such as solubility and/or uptake enhancer(s), and pH-lowering agent(s), are included in a sufficiently viscous protective syrup to permit protected passage of the components of the embodiment through the stomach.

Suitable enteric coatings may be applied, for example, to capsules after the active and other components of the invention have been loaded within the capsule. In other embodiments, enteric coating is coated on the outside of a tablet or coated on the outer surface of particles of active components which are then pressed into tablet form, or loaded into a capsule.

In some embodiments it may be desirable that all components of the invention be released from the carrier or vehicle, and solubilized in the intestinal environment as simultaneously as possible. It may also be preferred in some embodiments that the vehicle or carrier release the active components in the small intestine where uptake enhancers that increase transcellular or paracellular transport are less likely to cause undesirable side effects than if the same uptake enhancers were later released in the colon. It will be appreciated, however, that the present invention is believed effective in the colon as well as in the small intestine. Numerous vehicles or carriers, in addition to the ones discussed above, are known in the art.

In some embodiments, it may be desirable (especially in optimizing how simultaneously the components of the invention are released) to keep the amount of enteric coating low. In some embodiments, an enteric coating adds no more than 30% to the weight of the remainder of pharmaceutical composition such as a solid dosage form (the “remainder” being the pharmaceutical composition exclusive of enteric coating itself). In other embodiments, an enteric coating adds less than 20%, less than 19%, less than 18%, less than 17%, less than 16%, less than 15%, less than 14%, less than 13%, less than 12%, less than 11%, or less than 10%. In some embodiments, a protective vehicle such as an enteric coating constitutes an amount of or less than approximately 25%, 24%, 23%, 22%, 21%, 20%, 19%, 18%, 17%, 16%, 15%, 14%, 13%, 12%, 11%, 10%, 9%, 8%, 7%, 6%, 5% of the total weight of a pharmaceutical composition (e.g., a solid dosage form).

Routes of Administration

An angiotensin (1-7) peptide or angiotensin (1-7) receptor agonist as described herein (or a composition or medicament containing an angiotensin (1-7) peptide or Angiotensin (1-7) receptor agonist as described herein) may be administered by any appropriate route. In some embodiments, the angiotensin (1-7) peptide is administered parenterally. In some embodiments, the parenteral administration is selected from intravenous, intradermal, inhalation, transdermal (topical), intraocular, intramuscular, subcutaneous, intramuscular, and/or transmucosal administration. In some embodiments, an angiotensin (1-7) peptide or angiotensin (1-7) receptor agonist as described herein is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, thigh region, abdominal region, gluteal region, or scapular region. In some embodiments, an angiotensin (1-7) peptide or angiotensin (1-7) receptor agonist as described herein is administered intravenously. In other embodiments, an angiotensin (1-7) peptide or angiotensin (1-7) receptor agonist as described herein is administered by direct administration to a target tissue, such as heart or muscle (e.g., intramuscular), tumor (intratumorally), nervous system (e.g., direct injection into the brain; intraventricularly; intrathecally). Alternatively, an angiotensin (1-7) peptide or angiotensin (1-7) receptor agonist as described herein (or a composition or medicament containing an angiotensin (1-7) peptide or angiotensin (1-7) receptor agonist as described herein) can be administered by inhalation, parenterally, intradermally, transdermally, or transmucosally (e.g., orally or nasally). More than one route can be used concurrently, if desired.

In some embodiments, an angiotensin (1-7) peptide or angiotensin (1-7) receptor agonist as described herein is administered orally. In some embodiments, the present invention provides solid dosage forms of an angiotensin (1-7) peptide or angiotensin (1-7) receptor agonist as described herein for oral administration including (a) an angiotensin (1-7) peptide, (b) at least one pharmaceutically acceptable pH-lowering agent, (c) at least one absorption enhancer effective to promote bioavailability of the angiotensin (1-7) peptide, and (d) a protective vehicle. In some embodiments, the solid dosage form is a capsule or tablet. Various methods and ingredients for making oral formulations are known in the art and it is expected that one of skill would be able to determine which of these methods and ingredients will be compatible with the invention as described in this specification and/or in U.S. Provisional Patent Application Ser. No. 61/701,972, filed on Sep. 17, 2012, the disclosure of which is hereby incorporated in its entirety. Such methods and ingredients are also contemplated as within the scope of the present invention.

Dosing

In some embodiments, a composition is administered in a therapeutically effective amount and/or according to a dosing regimen that is correlated with a particular desired outcome (e.g., with treating or reducing risk for Muscular Dystrophy).

In some embodiments, the angiotensin (1-7) peptide is administered at an effective dose periodically at an administration interval such that at least one symptom or feature of a muscular dystrophy is reduced in intensity, severity, duration, or frequency or has delayed in onset. In some embodiments, the at least one symptom or feature of muscular dystrophy is selected from the group consisting of muscle wasting, muscle weakness, muscle fragility, muscle pseudohypertrophy, joint contracture, skeletal deformation, cardiomyopathy, impaired swallowing, impaired bowel and bladder function, muscle ischemia, cognitive impairment, behavioral dysfunction, socialization impairment, scoliosis, and impaired respiratory function.

In some embodiments, a formulation comprising an angiotensin (1-7) peptide or angiotensin (1-7) receptor agonist as described herein is administered as a single dose. In some embodiments, a formulation comprising an angiotensin (1-7) peptide or angiotensin (1-7) receptor agonist as described herein is administered at regular intervals. Administration at an “interval,” as used herein, indicates that the therapeutically effective amount is administered periodically (as distinguished from a one-time dose). The interval can be determined by standard clinical techniques. In some embodiments, a formulation comprising an angiotensin (1-7) peptide or angiotensin (1-7) receptor agonist as described herein is administered bimonthly, monthly, twice monthly, triweekly, biweekly, weekly, twice weekly, thrice weekly, daily, twice daily, or every six hours. The administration interval for a single individual need not be a fixed interval, but can be varied over time, depending on the needs of the individual.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day. In some embodiments, repeat doses are given at the same time of day (e.g. 10 am). In some embodiments, repeat doses are given at different times of day.

In some embodiments, a formulation comprising an angiotensin (1-7) peptide or angiotensin (1-7) receptor agonist as described herein is administered at regular intervals indefinitely. In some embodiments, a formulation comprising an angiotensin (1-7) peptide or angiotensin (1-7) receptor agonist as described herein is administered at regular intervals for a defined period. In some embodiments, a formulation comprising an angiotensin (1-7) peptide or angiotensin (1-7) receptor agonist as described herein is administered at regular intervals for 5 years, 4, years, 3, years, 2, years, 1 year, 11 months, 10 months, 9 months, 8 months, 7 months, 6 months, 5 months, 4 months, 3 months, 2 months, a month, 3 weeks, 2, weeks, a week, 6 days, 5 days, 4 days, 3 days, 2 days or a day.

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. For example, in some embodiments, an appropriate dose or amount is a dose or amount sufficient to increase muscle mass by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more. For example, in some embodiments, an appropriate dose or amount is a dose or amount sufficient to reduce incidence or severity of muscle ischemia by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100%. Alternatively or additionally, in some embodiments, an appropriate dose or amount is determined through use of one or more in vitro or in vivo assays to help identify desirable or optimal dosage ranges or amounts to be administered.

In various embodiments, an angiotensin (1-7) peptide or angiotensin (1-7) receptor agonist is administered at a therapeutically effective amount. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition). In some particular embodiments, appropriate doses or amounts to be administered may be extrapolated from dose-response curves derived from in vitro or animal model test systems.

Therapeutically effective dosage amounts of angiotensin (1-7) peptides or angiotensin (1-7) receptor agonists, including derivatives, analogs, and/or salts may be present in varying amounts in various embodiments. In some embodiments, a therapeutically effective dosage amount can be, for example, about 1-10,000 μg/kg, about 5-1,500 μg/kg, about 100-1,000 μg/kg, or 400-500 μg/kg. In some embodiments, the therapeutically effective dosage amount can be, for example, about 1 μg/kg, 2.5 μg/kg, 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 400 μg/kg, 500 μg/kg, 600 μg/kg, 700 μg/kg, 800 μg/kg, 900 μg/kg, 1000 μg/kg, or 1500 μg/kg. The effective dose for a particular individual can be varied (e.g., increased or decreased) over time, depending on the needs of the individual. In some embodiments, the therapeutically effective amount described herein is provided in one dose. In some embodiments, the therapeutically effective amount described herein is provided in one day.

In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight. In some embodiments, the therapeutically effective amount described herein is provided in one dose. In some embodiments, the therapeutically effective amount described herein is provided in one day.

In still other embodiments, a therapeutically effective dosage amount may be, for example, about 0.001 mg/kg weight to about 1 mg/kg weight, e.g. from about 0.001 mg/kg weight to about 0.9 mg/kg weight, from about 0.001 mg/kg weight to about 0.8 mg/kg weight, from about 0.001 mg/kg weight to about 0.8 mg/kg weight, from about 0.001 mg/kg weight to about 0.7 mg/kg weight, from about 0.001 mg/kg weight to about 0.6 mg/kg weight, from about 0.001 mg/kg weight to about 0.5 mg/kg weight, from about 0.01 mg/kg weight to about 1 mg/kg weight, from about 0.01 mg/kg weight to about 0.9 mg/kg weight, from about 0.01 mg/kg weight to about 0.8 mg/kg weight, from about 0.01 mg/kg weight to about 0.7 mg/kg weight, from about 0.01 mg/kg weight to about 0.6 mg/kg weight, from about 0.01 mg/kg weight to about 0.5 mg/kg weight, from about 0.02 mg/kg weight to about 1 mg/kg weight, from about 0.02 mg/kg weight to about 0.9 mg/kg weight, from about 0.02 mg/kg weight to about 0.8 mg/kg weight, from about 0.02 mg/kg weight to about 0.7 mg/kg weight, from about 0.02 mg/kg weight to about 0.6 mg/kg weight, from about 0.02 mg/kg weight to about 0.5 mg/kg weight, from about 0.03 mg/kg weight to about 1 mg/kg weight, from about 0.03 mg/kg weight to about 0.9 mg/kg weight, from about 0.03 mg/kg weight to about 0.8 mg/kg weight, from about 0.03 mg/kg weight to about 0.7 mg/kg weight, from about 0.03 mg/kg weight to about 0.6 mg/kg weight, from about 0.03 mg/kg weight to about 0.5 mg/kg weight, from about 0.04 mg/kg weight to about 1 mg/kg weight, from about 0.04 mg/kg weight to about 0.9 mg/kg weight, from about 0.04 mg/kg weight to about 0.8 mg/kg weight, from about 0.04 mg/kg weight to about 0.7 mg/kg weight, from about 0.04 mg/kg weight to about 0.6 mg/kg weight, from about 0.04 mg/kg weight to about 0.5 mg/kg weight, from about 0.05 mg/kg weight to about 1 mg/kg weight, from about 0.05 mg/kg weight to about 0.9 mg/kg weight, from about 0.05 mg/kg weight to about 0.8 mg/kg weight, from about 0.05 mg/kg weight to about 0.7 mg/kg weight, from about 0.05 mg/kg weight to about 0.6 mg/kg weight, from about 0.05 mg/kg weight to about 0.5 mg/kg weight. In some embodiments, the therapeutically effective amount described herein is provided in one dose. In some embodiments, the therapeutically effective amount described herein is provided in one day.

In still other embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual. In some embodiments, the therapeutically effective amount described herein is provided in one dose. In some embodiments, the therapeutically effective amount described herein is provided in one day.

In some embodiments, the angiotensin (1-7) peptide is administered at an effective dose ranging from about 1-1,000 μg/kg/day (e.g., ranging from about 1-900 μg/kg/day, 1-800 μg/kg/day, 1-700 μg/kg/day, 1-600 μg/kg/day, 1-500 μg/kg/day, 1-400 μg/kg/day, 1-300 μg/kg/day, 1-200 μg/kg/day, 1-100 μg/kg/day, 1-90 μg/kg/day, 1-80 μg/kg/day, 1-70 μg/kg/day, 1-60 μg/kg/day, 1-50 μg/kg/day, 1-40 μg/kg/day, 1-30 μg/kg/day, 1-20 μg/kg/day, 1-10 μg/kg/day). In some embodiments, the angiotensin (1-7) peptide is administered at an effective dose ranging from about 1-500 μg/kg/day. In some embodiments, the angiotensin (1-7) peptide is administered at an effective dose ranging from about 400-500 μg/kg/day. In some embodiments, the angiotensin (1-7) peptide is administered at an effective dose ranging from about 1-100 μg/kg/day. In some embodiments, the angiotensin (1-7) peptide is administered at an effective dose ranging from about 1-60 μg/kg/day. In some embodiments, the angiotensin (1-7) peptide is administered at an effective dose selected from about 1, 2, 4, 6, 8, 10, 15, 20, 25, 30, 35, 40, 45, 50, 75, 100, 150, 200, 250, 300, 350, 400, 450, 500, 550, 600, 650, 700, 750, 800, 850, 900, 950, or 1,000 μg/kg/day.

In some embodiments, a provided composition is provided as a pharmaceutical formulation. In some embodiments, a pharmaceutical formulation is or comprises a unit dose amount for administration in accordance with a dosing regimen correlated with achievement of the reduction in intensity, severity, duration, frequency, or delay in onset of at least one symptom or feature of muscular dystrophy.

Combination Therapy

In some embodiments, an angiotensin (1-7) peptide and/or an angiotensin (1-7) receptor agonist is administered in combination with one or more known therapeutic agents (e.g. anti-muscular dystrophy medications) currently used for muscular dystrophy prophylaxis and/or treatment. In some embodiments, the known therapeutic agent(s) is/arc administered according to its standard or approved dosing regimen and/or schedule. In some embodiments, the known therapeutic agent(s) is/are administered according to a regimen that is altered as compared with its standard or approved dosing regimen and/or schedule. In some embodiments, such an altered regimen differs from the standard or approved dosing regimen in that one or more unit doses is altered (e.g., reduced or increased) in amount, and/or in that dosing is altered in frequency (e.g., in that one or more intervals between unit doses is expanded, resulting in lower frequency, or is reduced, resulting in higher frequency).

In some embodiments, the angiotensin (1-7) peptide is administered in combination with one or more anti-muscular dystrophy medications. In some embodiments, the one or more anti-muscular dystrophy medications is a glucocorticoids (e.g. prednisone or VBP15), a phosphodiesterase type 5 (pde5) inhibitor, or other therapy such as nitric oxide boosting medications (e.g. HCT 1026 or NCX 320), drisapersen, anti-sense oligonucleotides (e.g. AVI-4658), and others. In some embodiments, the one or more anti-muscular dystrophy medications is selected from the group consisting of Eteplirsen (AVT-4658), HCT 1026, NCX 320 sildenafil, tadalafil, vardenafil, avanafil, iodenafil, mirodenafil, udenafil, zaprinast, a corticosteroid, and combinations thereof. In some embodiments, the angiotensin (1-7) peptide and/or angiotensin (1-7) receptor agonist is administered simultaneously with one or more anti-muscular dystrophy medications. In some embodiments, the angiotensin (1-7) peptide and/or angiotensin (1-7) receptor agonist and the one or more anti-muscular dystrophy medications are administered sequentially.

### Kits

The present invention further provides kits or other articles of manufacture which contains an angiotensin (1-7) peptide or an angiotensin (1-7) receptor agonist, or a formulation containing the same, and provides instructions for its reconstitution (if lyophilized) and/or use. Kits or other articles of manufacture may include a container, a syringe, vial and any other articles, devices or equipment useful in administration (e.g., subcutaneous, oral, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, the container may hold formulations and a label on, or associated with, the container that may indicate directions for reconstitution and/or use. For example, the label may indicate that the formulation is reconstituted to concentrations as described above. The label may further indicate that the formulation is useful or intended for, for example, subcutaneous administration. In some embodiments, a container may contain a single dose of a stable formulation containing an angiotensin (1-7) peptide. In various embodiments, a single dose of the stable formulation is present in a volume of less than about 15 ml, 10 ml, 5.0 ml, 4.0 ml, 3.5 ml, 3.0 ml, 2.5 ml, 2.0 ml, 1.5 ml, 1.0 ml, or 0.5 ml. Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation will generally be at least 1 mg/ml (e.g., at least 5 mg/ml, at least 10 mg/ml, at least 20 mg/ml, at least 30 mg/ml, at least 40 mg/ml, at least 50 mg/ml, at least 75 mg/ml, at least 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

### EXAMPLES

### Example 1—Administration of Angiotensin (1-7), PanCyte, or Linear PanCyte in the mdx Mouse Model of Duchenne Muscular Dystrophy

In this Example, mdx mice, a known and accepted model of Duchenne Muscular Dystrophy (DMD) are used to assess the effects of several angiotensin (1-7) peptides and an angiotensin (1-7) receptor agonist, AVE0991, on the muscle degeneration typically observed in DMD patients. See, Dangain and Vrbova, Muscle development in mdx mutant mice, 1984, Muscle Nerve 7: 700-704; see also Tanabe et al., Skeletal muscle pathology in X-chromosome-linked muscular dystrophy (mdx) mouse, 1986, Acta Neuropathol, 69:91-95; Kobayashi et al., Endpoint measures in the mdx mouse relevant for muscular dystrophy pre-clinical studies, 2012, Acta Materialia, 22: 34-42. Specifically, the mdx mouse has a point mutation within its dystrophin gene, which leads to disruption of dystrophin production and results in almost no functional dystrophin being present in the mouse. The lack of functional dytrophin eventually leads to the formation of significant fibrosis and significant weakness across many muscles including the diaphragm, typically resulting in death.

In this example, three angiotensin (1-7) peptides, namely, angiotensin (1-7) (SEQ ID NO:1), PanCyte (SEQ ID NO:22), and Linear PanCyte (also referred to as “TXA301”; SEQ ID NO: 2) and a small molecule angiotensin (1-7) receptor agonist AVE0991 are used to examine their effect on the progression of muscle wasting over time. Specifically, 110 mdx mice, (10 per group) are placed into one of the groups outlined in Table 1 below.

TABLE 1
Study Design

| | | | | | Route of | Dosing |
|---|---|---|---|---|---|---|
| Group | Agent | Dose | | N | Admin. | Frequency |
| 1 | Vehicle | PBS | | 10 | Subcutaneous | Daily |
| | Control | | | | | |
| | (PBS) | | | | | |
| 2 | Ang (1-7) | 50 | μg/kg/day | 10 | Subcutaneous | Daily |
| 3 | Ang (1-7) | 500 | μg/kg/day | 10 | Subcutaneous | Daily |
| 4 | Ang (1-7) | 1,000 | μg/kg/day | 10 | Subcutaneous | Daily |
| 5 | PanCyte | 50 | μg/kg/day | 10 | Subcutaneous | Daily |
| 6 | PanCyte | 500 | μg/kg/day | 10 | Subcutaneous | Daily |
| 7 | PanCyte | 1,000 | μg/kg/day | 10 | Subcutaneous | Daily |
| 8 | Linear | 50 | μg/kg/day | 10 | Subcutaneous | Daily |
| | PanCyte | | | | | |
| 9 | Linear | 500 | μg/kg/day | 10 | Subcutaneous | Daily |
| | PanCyte | | | | | |
| 10 | Linear | 1,000 | μg/kg/day | 10 | Subcutaneous | Daily |
| | PanCyte | | | | | |
| 11 | AVE0991 | 300 | μg/kg/day | 10 | Subcutaneous | Daily |

In this example, linear PanCyte, PanCyte, and AVE0991 are prepared in saline and administrated subcutaneously via subscapular injection at a dose volume of 100 μl/mouse daily for twenty-eight days. Dosing solutions are prepared fresh every three days.

One week after the final injection, each animal is sacrificed, and several muscles are removed and weighed to determine if administration of an angiotensin (1-7) peptide or angiotensin (1-7) receptor agonist is sufficient to increase the muscle mass of treated mdx mice as compared to control animals. In this study, the muscles examined are: the diaphragm, gastrocnemius, quadriceps, and triceps.

Myofiber size is also determined in both treated and control mice using standard histological and immunohistochemical methods. Histopathology according to known methods (e.g., Hematoxylin & Eosin staining) are also used to determine if the muscle mass is comprised of healthy fibers. Indicators of healthy muscle tissue include the presence of rounded fibers and centralized nuclei. Exemplary methods of analyzing muscle tissue include those described in: Meola G, “Advanced microscopic and histochemical techniques: Diagnostic tools in the molecular era of myology,” 2005, Eur J Histochem 49(1):93-96, and Karpati et al., “Tracer and marker techniques in the microscopic study of skeletal muscles,” 1981, Methods Achiev Exp Pathol, 10:101-137, the disclosures of which are hereby incorporated by reference.

It is expected that administration of an angiotensin (1-7) peptide increases muscle mass in mdx mice. Hypertrophy will be observed in various tissues. Administration of an angiotensin (1-7) peptide will also increase healthy muscle tissue in mdx mice.

### Example 2—Administration of Ang (1-7) in the Sgcd−/− Mouse Model of Muscular Dystrophy

In this Example, Sgcd −/− mice, a known model of limb-girdle muscular dystrophy, were used to assess the effects of angiotensin (1-7) peptides on skeletal muscle fibrosis, blood pressure, heart rate, cardiac activity, baroreflex sensitivity, oxidative stress, AT₁R receptor expression, and resting cardiac and vagal sympathetic tone. Sgcd −/− mice are missing a key component of the sarcoglycan complex, specifically, they are missing a link between the F-actin cytoskeleton and the extracellular matrix. This defect causes muscle wasting and weakness due to a reduction in muscle fiber integrity and dysregulation in cell signaling in muscle cells. It has been previously shown that this defect results in a reduction of locomotor activity and autonomic dysfunction at a young age. The autonomic dysfunction is thought to lead to cardiac dysfunction later in life.

To examine the effects of angiotensin (1-7) peptides on disruption of normal motor activity in Sgcd −/− mice, conscious, young (10-15 wks) Sgcd−/− and control C57BL6 mice were analyzed by radio-telemetry. Radio-telemeters were implanted into the aorta via the left carotid artery under ketamine-xylazine anesthesia when mice were 10 weeks of age. Following recovery, blood pressure, heart rate and locomotor activity were measured 24 hours/day. Blood pressure measurements were sampled at 2000 Hz for 1 hour periods to enable beat-to-beat measurements of systolic arterial pressure and pulse interval (PI). Baroreflex control of heart rate was assessed from spontaneous fluctuations in systolic blood pressure and pulse interval using known sequence techniques.

Baroreflex sensitivity was calculated from spontaneous fluctuations in systolic blood pressure and pulse interval using the sequence technique. Specifically, baroreflex sensitivity was calculated as the average slope of sequences of 4 or more consecutive blood pressure pulses where the change in blood pressure and pulse interval are positively correlated (r²>0.85).

Cardiac sympathetic tone and parasympathetic tone were calculated from heart rate responses to the β-adrenergic receptor blocker propranolol and the muscarinic cholinergic receptor blocker methylatropine, respectively. Specifically, resting cardiac sympathetic and parasympathetic (vagal) tone was estimated from heart rate responses to propranolol (1 μg/g, IP) and methylatropine (1 μg/g, IP), respectively. Vasomotor sympathetic tone was calculated from the decrease in mean arterial blood pressure in response to injection of the ganglionic blocker chlorisondamine (12 μg/g, IP).

Osmotic minipumps (Alzet) were implanted with either Ang (1-7) (also referred to as “TXA127”) or vehicle in mice pups at 3 weeks of age, under ketamine-xylazine anesthesia.

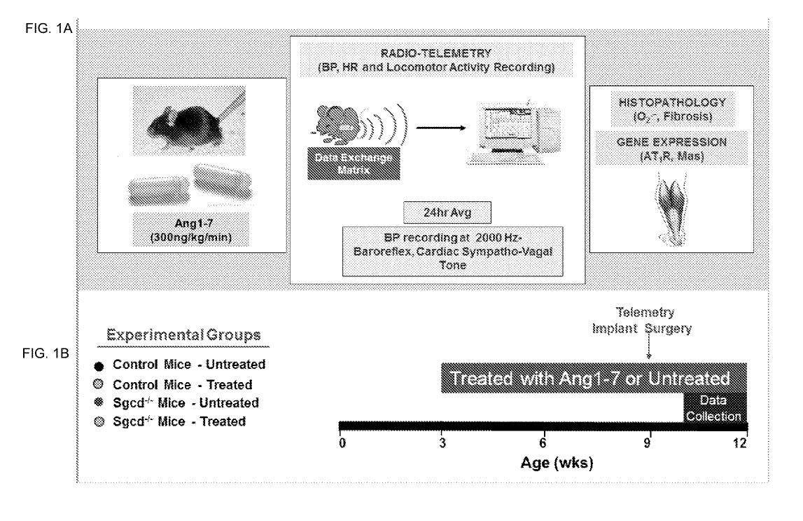

Data were collected over 2-3 days, and 24 hour averages were calculated. Separate groups of control and Sgcd−/− mice were infused with Ang-(1-7) (300 ng/kg/min) for 8 weeks beginning at 3 weeks of age. Measurements were obtained in these mice using the same protocols as described above. FIG. 1A shows depictions of some of the data collection techniques applied in this Example and FIG. 1B shows the experimental for this study.

To assess skeletal muscle phenotypes, paraffin sections of skeletal muscle (quadriceps) were stained with Masson Trichrome to assess histopathology and fibrosis. Frozen sections of skeletal muscle were incubated with polyclonal anti-δ and anti-β sarcoglycan primary antibody, a nuclear stain, and Alexa Fluor 488. AT₁R (Ang type 1 receptor) and Mas (Ang(1-7) receptor) were detected with rabbit anti-AT₁R, and anti-Mas nuclear stains, and secondary antibody goat anti-rabbit IgG. Oxidative stress (O2.⁻) was measured by dihydrocthidium (DHE) fluorescence.

Data in this example are presented as ±SE. Results were analyzed by ANOVA, and paired and unpaired t-tests as appropriate, significance was determined at p<0.05.

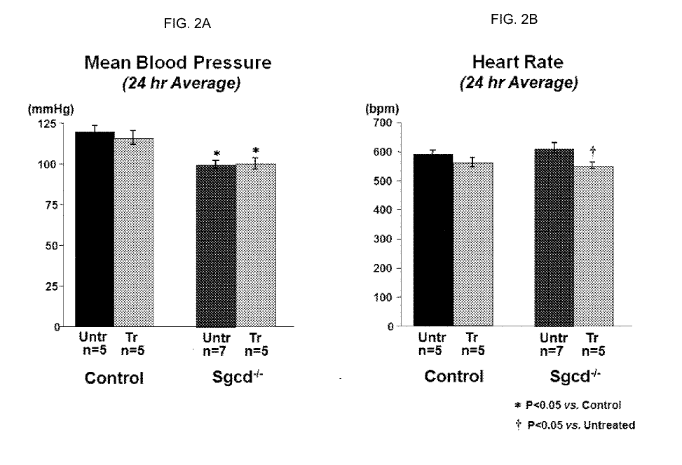

FIGS. 2A and 2B show the levels of mean arterial blood pressure and heart rate in untreated and Ang (1-7)-treated control and Sgcd −/− mice. Panel A shows that the lower blood pressure typically observed in Sgcd −/− mice was not significantly affected by Ang (1-7) treatment. Panel B shows that Ang (1-7) treatment of Sgcd −/− mice appears to mildly decrease the heart rate of animals versus untreated Sgcd −/− controls.

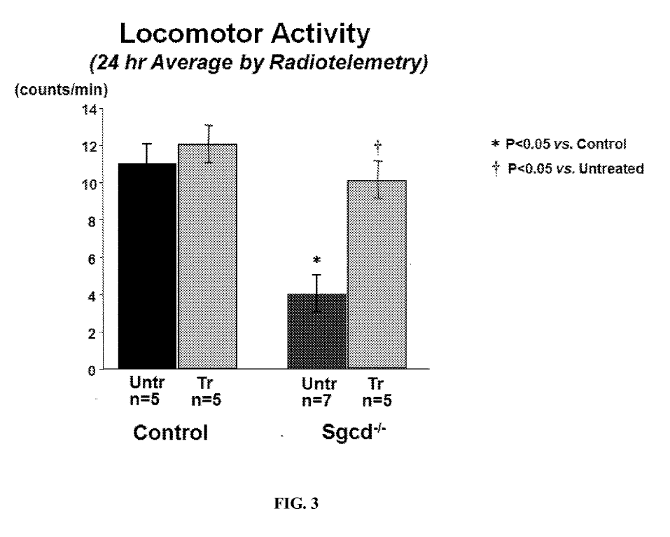

As shown in FIG. 3, locomotor activity was significantly reduced in Sgcd −/− mice, as expected. However, this reduction in locomotor activity was drastically reduced by treatment with Ang (1-7). In fact, the levels of locomotion in treated Sgcd −/− mice did not vary significantly from Control C57BL6 mice.

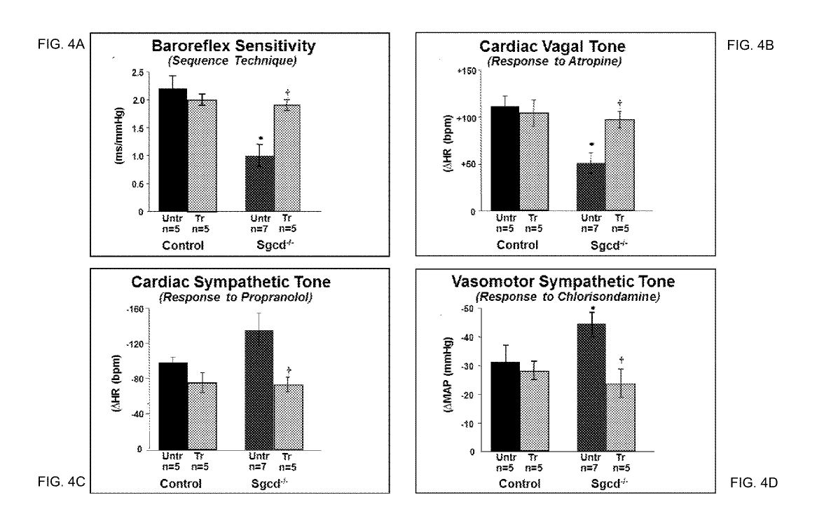

FIG. 4 shows a comparison of 4A) baroreflex sensitivity, 4B) cardiac vagal tone, 4C) cardiac sympathetic tone, and 4D) vasomotor sympathetic tone, between control and Sgcd −/− mice as well as between untreated and Ang (1-7)-treated animals. As shown in panels A and B, the decreases in baroreflex sensitivity and cardiac vagal tone in Sgcd −/− mice, as compared to normal mice, were essentially abrogated by treatment with Ang (1-7). Panels C and D show that the increases in cardiac sympathetic tone and vasomotor sympathetic tone typically observed in Sgcd −/− mice were also essentially abrogated by treatment with Ang (1-7).

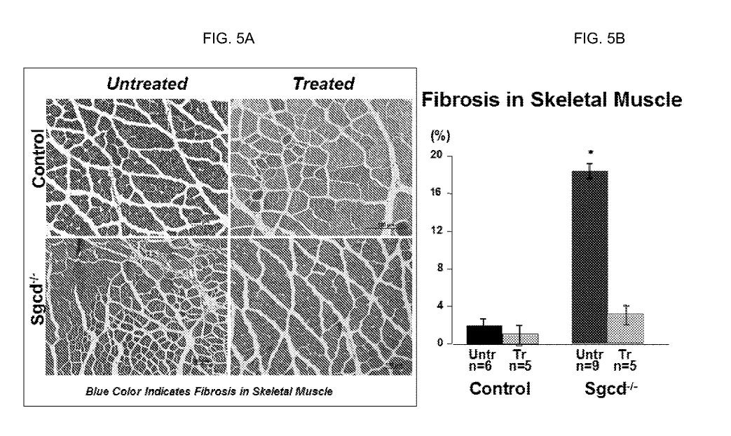

FIG. 5 shows both histopathological (panel 5A) and quantitative (panel 5B) data regarding fibrosis of skeletal muscle, here, the quadriceps. As can be seen in both panels, the significant fibrosis observed in Sgcd −/− mice was markedly reduced by treatment with Ang (1-7). In fact, panel 5B shows that the levels of fibrosis observed in Sgcd −/− mice treated with Ang (1-7) were comparable to control C57BL6 mice.

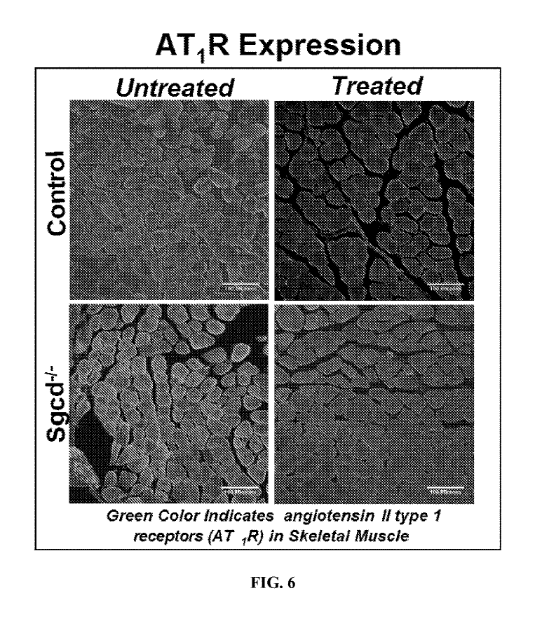

In Sgcd −/− mice, an increase in angiotensin type 1 receptors (AT₁R) is observed and correlated with decreased locomotor activity and autonomic dysfunction (see Sabharwal et al, Receptor activity-modifying protein 1 increases baroreflex sensitivity and attenuates Angiotensin-induced hypertension, 2010, Hypertension, 55(3): 627-635). FIG. 6 shows that treatment with Ang (1-7) essentially abrogates the increase in AT₁R observed in the untreated Sgcd −/− mice.

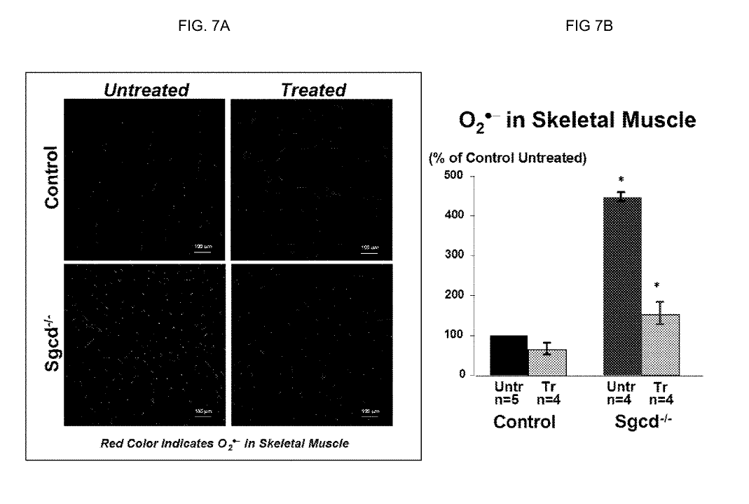

Increased levels of oxidative stress, such as through increased levels of superoxide, have been correlated with a wide variety of symptoms of muscular dystrophy (see Terrill et al., Oxidative stress and pathology in muscular dystrophies: focus on protein thiol oxidation and dysferlinopathies, 2013, FEBS Journal doi: 10.1111/febs. 12142; see also Murphy and Kehrer, Oxidative stress and muscular dystrophy, 1989, Chem Biol Interact, 69(2-3): 101-173). A similar phenomenon is typically observed in Sgcd −/− mice and this is thought to correlate with the difficulties in locomotor activity and autonomic function observed in this model. FIGS. 7A and 7B shows that the dramatically increased levels of superoxide observed in untreated Sgcd −/− mice was strongly attenuated by treatment with Ang (1-7). Panel A shows dihydroethidium fluorescence of sample, while panel B shows a quantitative assessment of the levels of superoxide in each group.

The ability of Ang(1-7) peptides to reduce skeletal muscle fibrosis and restore locomotor activity and sympathovagal balance in Sgcd −/− mice, without lowering blood pressure shows that Ang(1-7) peptides have significant therapeutic potential as treatments for muscular dystrophy. As shown in this Example, Ang (1-7) peptides improved every observed measure of dysfunction in Sgcd −/− mice, as compared to untreated Sgcd −/− mice. Based on this discovery, as illustrated by the above examples, administration of angiotensin (1-7) peptides and/or angiotensin (1-7) receptor agonists may provide a powerful and novel approach to the treatment of muscular dystrophy.

### Example 3—Oral Administration of PanCyte and Linear PanCyte Compositions to mdx Mice

In this example, specific angiotensin (1-7) peptides, namely PanCyte (SEQ ID NO: 22) and Linear PanCyte (also referred to as “TXA301”; SEQ ID NO: 2) are used in exemplary oral formulations to assess the effect of oral administration on the condition of mdx mice. In this Example, oral gavage is used to administer agent according to known methods, and separate aqueous formulations of PanCyte and Linear PanCyte are each made with citric acid, laurolyl carnitine, and hydroxypropyl methylcellulose phthalate according to methods described in U.S. Provisional Patent Application 61/701,972, filed Sep. 17, 2012.

A total of 80 mdx mice separated into eight groups, including a PBS control and a naked Angiotensin (1-7) peptide control, are used in this example according the design shown in Table 2:

TABLE 2
Study Design

| | | | | | Route of | Dosing |
|---|---|---|---|---|---|---|
| Group | Agent | Dose | | N | Admin. | Frequency |
| 1 | Vehicle | PBS | | 10 | Oral | Daily |
| | Control | | | | | |
| | (PBS) | | | | | |
| 2 | Angiotensin | 50 | μg/kg/day | 10 | Oral | Daily |
| | (1-7) | | | | | |
| 3 | PanCyte | 50 | μg/kg/day | 10 | Oral | Daily |
| | Composition | | | | | |
| 4 | PanCyte | 500 | μg/kg/day | 10 | Oral | Daily |
| | Composition | | | | | |
| 5 | PanCyte | 1,000 | μg/kg/day | 10 | Oral | Daily |
| | Composition | | | | | |
| 6 | Linear | 50 | μg/kg/day | 10 | Oral | Daily |
| | PanCyte | | | | | |
| | Composition | | | | | |
| 7 | Linear | 500 | μg/kg/day | 10 | Oral | Daily |
| | PanCyte | | | | | |
| | Composition | | | | | |
| 8 | Linear | 1,000 | μg/kg/day | 10 | Oral | Daily |
| | PanCyte | | | | | |
| | Composition | | | | | |

As in Example 1, one week after the final administration, each animal is sacrificed, and several muscles are removed and weighed to determine if oral administration of an angiotensin (1-7) peptide and/or a composition including either PanCytc or Linear PanCytc is sufficient to increase the muscle mass of treated mdx mice as compared to control animals. In this study, the muscles examined are: the diaphragm, gastrocnemius, quadriceps, and triceps.

Myofiber size is also determined in both treated and control mice using standard histological and immunohistochemical methods. Histopathology according to known methods (e.g., Hematoxylin & Eosin staining) are also used to determine if the muscle mass is comprised of healthy fibers as described above in Example 1. Indicators of healthy muscle tissue include the presence of rounded fibers and centralized nuclei.

It is expected that oral administration of a composition including either PanCyte or Linear PanCyte will be sufficient to increase muscle mass in mdx mice. Hypertrophy will be observed in various tissues. Oral administration of a composition including either PanCyte or Linear PanCyte will also increase healthy muscle tissue in mdx mice.

### Example 4—Functional Performance and Degree of Fibrosis in the Sgcd−/− Mouse Model of Muscular Dystrophy after Treatment with and Angiotensin (1-7) Peptide

In this Example, Sarcoglycan delta deficient mice (Sgcd−/−), an established mouse model of Limb Girdle Muscular Dystrophy-2F (LGMD-2F), was used to examine the effects of angiotensin (1-7) peptides on the progression of functional impairment and fibrosis in sufferers of the disease. It is generally known that Sgcd−/− mice exhibit reduced locomotor activity and dystrophic phenotype in skeletal muscles at a young age, and this Example shows that treatment with Angiotensin (1-7) peptides improves locomotor activity and decreases the rate of fibrosis.

A total of 32 Sgcd −/− mice were randomly assigned to one of four treatment groups (n=8 in each): a saline group (vehicle) a TXA127 group, a Pancyte group, and a TXA301 group. The doses of Angiotensin (1-7) peptides administered was 500 μg/kg/day for TXA127, 50 μg/kg/day for Pancyte, and 50 μg/kg/day for TXA301. Osmotic minipumps containing the treatments were implanted in mice at 28-35 weeks of age, and the angiotensin (1-7) peptides were infused for 8 weeks. For the last three weeks of treatment, the mice were put in cages with running wheels attached to calculate the amount of voluntary exercise performed by each animal. Total number of wheel revolutions (distance ran) were collected daily. At the end of 8 weeks of treatment, quadriceps muscles were harvested from the mice for histopathological and oxidative stress analyses.

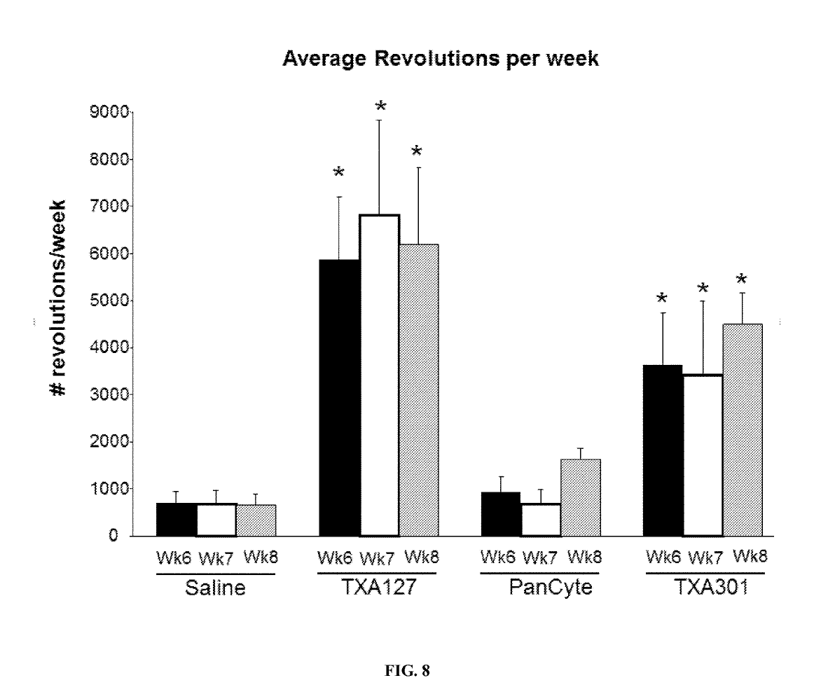

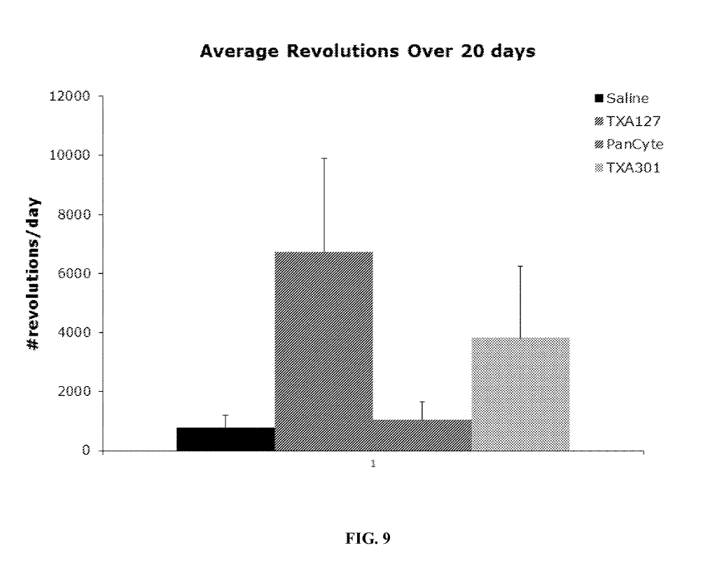

FIG. 8 shows the average revolutions made by mice in each treatment group in the running wheels during weeks 6, 7 and 8. Treatment with both TXA127 and TXA301 resulted in significant improvement in the amount of voluntary exercise performed as compared to mice in the saline control group, with TXA127 showing the greatest degree of improvement. FIG. 9 shows the same data as FIG. 8, only depicted as the accumulated totals per treatment group over the three week test period (weeks 6, 7, and 8). Again, treatment with both TXA127 and TXA301 showed significant increases in the amount of exercise performed by those groups as compared to saline control animals. Of note, treatment with TXA127 resulted in more than six fold improvement as compared to saline control animals.

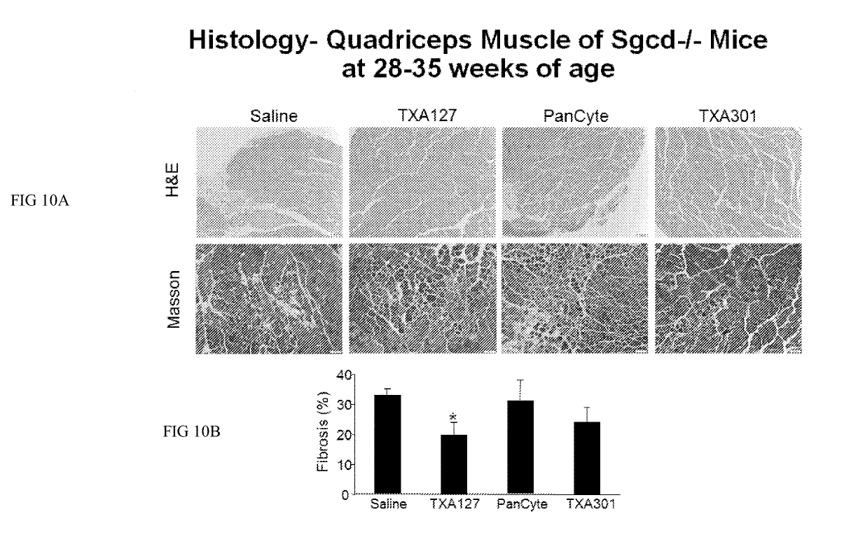

An H&E stain was used to examine the dystrophic morphology of the quadriceps muscles of the mice. FIG. 10A shows exemplary H&E (top row) and Masson (bottom row) stains from treated mice. As shown in FIGS. 10A and 10B, saline-treated Sgcd−/− mice exhibit increased centralized nuclei, fatty acid infiltration, and collagen deposition, while treatment with TXA127 significantly reduced fibrosis in skeletal muscle of Sgcd−/− mice. Additionally, mice treated with TXA301 also showed some improvement, while no reduction in fibrosis was observed in PanCyte-treated mice.

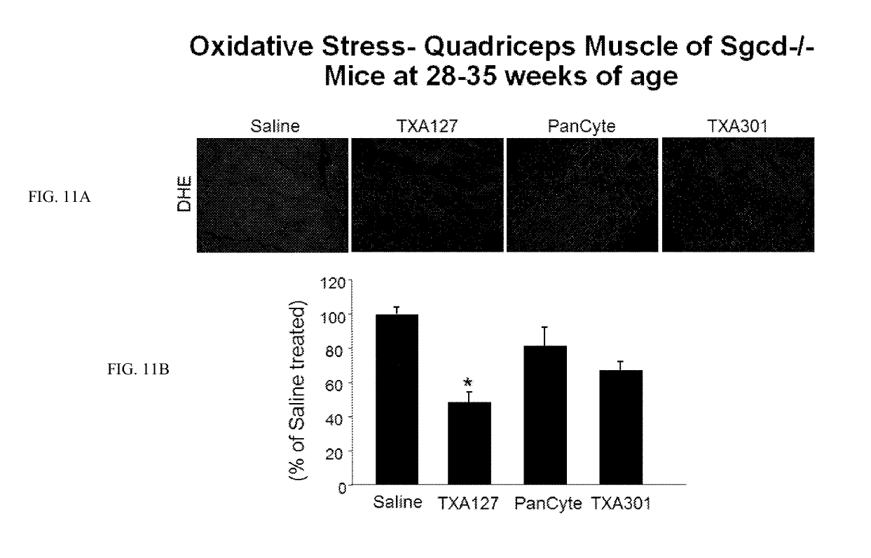

Dihydroethidium (DHE) was used to examine the degree of oxidative stress in treated Sgcd−/− mice. Briefly, DHE solution was applied to skeletal muscle sections and images were captured on a confocal microscope (red color is indicative of oxidative stress). Exemplary confocal images are shown in FIG. 11A. For quantification of degree of fibrosis, an average of six-sections of skeletal quadriceps muscle were obtained from each mouse. An intensity of fluorescence for each mouse was quantified using NIH ImageJ software and normalized to a percentage of the average fluorescence intensity measured in saline-treated Sgcd−/− mice. As shown in FIGS. 11A and 11B, saline-treated Sgcd−/− mice exhibit increased oxidative stress, which was significantly attenuated with TXA127.

This Example clearly shows that treatment with angiotensin (1-7) peptides, such as TXA127 and/or TXA301, has a significant effect in treating muscular dystrophy including, in this Example, symptoms such as degree of fibrosis, degree of oxidative stress, and degree of functional muscular impairment.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating a muscular dystrophy comprising administering to a subject suffering from or susceptible to a muscular dystrophy an angiotensin (1-7) peptide.

2. The method of claim 1, wherein the angiotensin (1-7) peptide is administered at an effective dose periodically at an administration interval such that at least one symptom or feature of a muscular dystrophy is reduced in intensity, severity, duration, or frequency or has delayed in onset.

3. The method of claim 2, wherein the at least one symptom or feature of muscular dystrophy is selected from the group consisting of muscle wasting, muscle weakness, muscle fragility, joint contracture, skeletal deformation, cardiomyopathy, impaired swallowing, muscle pseudohypertrophy, impaired bowel and bladder function, muscle ischemia, cognitive impairment, behavioral dysfunction, socialization impairment, scoliosis, and impaired respiratory function.

4. The method of claim 1, wherein the administration of the angiotensin (1-7) peptide results in muscle regeneration, fibrosis reduction, increased muscle strength, increased flexibility, increased range of motion, increased stamina, reduced fatiguability, increased blood flow, improved cognition, improved pulmonary function, and/or inflammation inhibition.

5. The method of claim 1, wherein the muscular dystrophy is selected from the group consisting of: Duchenne muscular dystrophy, Becker's muscular dystrophy, Emery-Dreifuss muscular dystrophy, limb-girdle muscular dystrophy, Miyoshi myopathy, congenital muscular dystrophy, facioscapulohumeral muscular dystrophy, oculopharyngeal muscular dystrophy, primary lateral sclerosis, spinal muscular atrophy, polymyositis, Guillian-Barre Syndrome, and myotonic muscular dystrophy.

6. The method of claim 5, wherein the congenital muscular dystrophy is selected from the group consisting of: laminin-α2-deficient congenital muscular dystrophy, Ullrich congenital muscular dystrophy, Walker-Warburg syndrome, Fukuyama congenital muscular dystrophy, and congenital muscular dystrophy with mental retardation and pachygyria.

7. The method of claim 1, wherein the angiotensin (1-7) peptide is administered parenterally.

8. The method of claim 7, wherein the parenteral administration is selected from intravenous, intradermal, inhalation, transdermal (topical), intraocular, intramuscular, subcutaneous, intramuscular, and/or transmucosal administration.

9. The method of claim 1, wherein the angiotensin (1-7) peptide is administered orally.

10. The method of claim 1, wherein the angiotensin (1-7) peptide is administered monthly, weekly, daily, or at variable intervals.

11. The method of claim 1, wherein the angiotensin (1-7) peptide is administered at an effective dose ranging from about 1-1,000 μg/kg/day.

12-13. (canceled)

14. The method of claim 1, wherein the angiotensin (1-7) peptide is administered in combination with one or more anti-muscular dystrophy medications.

15. The method of claim 14, wherein the one or more anti-muscular dystrophy medications is selected from the group consisting of Eteplirsen (AVI-4658), HCT 1026, NCX 320, sildenafil, tadalafil, vardenafil, avanafil, iodenafil, mirodenafil, udenafil, zaprinast, a corticosteroid, and combinations thereof.

16. The method of claim 1, wherein the angiotensin (1-7) peptide comprises the naturally-occurring Angiotensin (1-7) amino acid sequence of Asp¹-Arg²-Val³-Tyr4-Ile⁵-His⁶-Pro⁷(SEQ ID NO:1).

17. The method of claim 1, wherein the angiotensin (1-7) peptide is a functional equivalent of SEQ ID NO:1.

18. The method of claim 17, wherein the functional equivalent is a linear peptide.

19. The method of claim 18, wherein the linear peptide comprises a sequence that includes at least four amino acids from the seven amino acids that appear in the naturally-occurring Angiotensin (1-7), wherein the at least four amino acids maintain their relative positions as they appear in the naturally-occurring Angiotensin (1-7).

20. The method of claim 18, wherein the linear peptide contains 4-25 amino acids.

21. The method of claim 18, wherein the linear peptide is a fragment of the naturally-occurring Angiotensin (1-7).

22. The method of claim 18, wherein the linear peptide contains amino acid substitutions, deletions and/or insertions in the naturally-occurring Angiotensin (1-7).

23. The method of claim 22, wherein the linear peptide has an amino acid sequence of Asp¹-Arg²-Val³-Ser⁴-Ile⁵-His⁶-Cys⁷ (SEQ ID NO:2).

24. The method of claim 17, wherein the functional equivalent is a cyclic peptide.

25. The method of claim 24, wherein the cyclic peptide comprises a linkage between amino acids.

26. The method of claim 25, wherein the linkage is located at residues corresponding to positions Tyr⁴ and Pro⁷ in naturally-occurring Angiotensin (1-7).

27. The method of claim 25, wherein the linkage is a thioether bridge.

28. The method of claim 24, wherein the cyclic peptide comprises an amino acid sequence otherwise identical to the naturally-occurring Angiotensin (1-7) amino acid sequence of Asp¹-Arg²-Val³-Tyr⁴-Ile⁵-His⁶-Pro⁷ (SEQ ID NO:1).

29. The method of claim 24, wherein the cyclic peptide is a 4, 7-cyclized angiotensin (1-7) with the following formula:

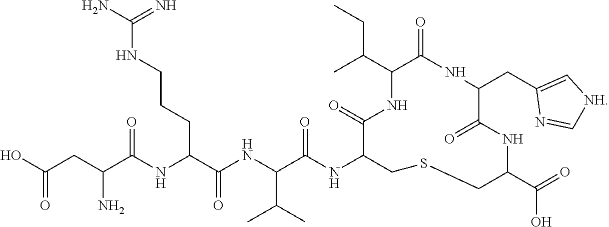 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

30. The method of claim 17, wherein the angiotensin (1-7) peptide comprises one or more chemical modifications to increase protease resistance, serum stability and/or bioavailability.

31. The method of claim 30, wherein the one or more chemical modifications comprise pegylation.

32. A method of treating muscular dystrophy comprising administering to a subject who is suffering from or susceptible to muscular dystrophy an angiotensin (1-7) receptor agonist.

33. The method of claim 32, wherein the angiotensin (1-7) receptor agonist is a non-peptidic agonist.

34. The method of claim 33, wherein the non-peptidic agonist is a compound with the following structure: 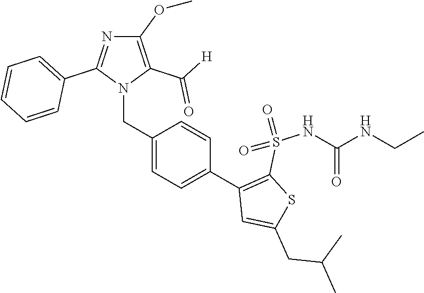 [SMILES: CCNC(=O)NS(=O)(=O)c1sc(CC(C)C)cc1-c1ccc(Cn2c(-c3ccccc3)nc(OC)c2C=O)cc1]
or a pharmaceutically acceptable salt thereof.

35. The method of any one of claim 32, wherein the angiotensin (1-7) receptor agonist is administered orally.
